(19) United States
(12) Patent Application Publication
(10) Pub. No.: US 2025/0221930 A1
(43) Pub. Date: Jul. 10, 2025

(54) A COMPOSITION COMPRISING LIPID NANOPARTICLES AND AN ANTIFUNGAL POLYENE

(71) Applicants: INSTITUT NATIONAL DE LA SANTÉ ET DE LA RECHERCHE MÉDICALE, Paris (FR); UNIVERSITE DE POITIERS, Poitiers (FR)

(72) Inventors: Frédéric TEWES, Poitiers (FR); Kévin BRUNET, Poitiers (FR)

(21) Appl. No.: 18/727,787

(22) PCT Filed: Jan. 10, 2023
(86) PCT No.: PCT/EP2023/050486

§ 371 Date: Jul. 10, 2024

(30) Foreign Application Priority Data
Jan. 11, 2022 (EP) 22305022.0
Oct. 28, 2022 (EP) 22306630.9

(51) Int. Cl. A61K 9/1271; A61K 9/107; A61K 9/51; A61K 31/4196; A61K 31/496; A61K 31/506; A61K 31/7048; A61P 31/10

(52) U.S. Cl. CPC A61K 9/1271; A61K 9/1075; A61K 9/5123; A61K 9/5146; A61K 31/4196; A61K 31/496; A61K 31/506; A61K 31/7048; A61P 31/10

(57) ABSTRACT

The present invention relates to a composition comprising an aqueous phase, a polyene antifungal and lipid nanoparticles which are dispersed in the aqueous phase, said lipid nanoparticles comprising an oily internal phase and an envelope, the envelope comprising a polyglycol ester of a hydroxy fatty acid, the polyene antifungal being dispersed in the aqueous phase and/or being part of the lipid nanoparticles. It further relates to lipid nanoparticles comprising an antifungal polyene, an oily internal phase and an envelope, the envelope comprising a polyglycol ester of a hydroxy fatty acid and the polyene antifungal being part of the lipid nanoparticles, and preparation method thereof. It also relates to the composition or lipid nanoparticles of the invention for use in the prevention or treatment of an infection caused by a fungus, preferably a fungus of the Mucorales order.

### INTRODUCTION/FIELD OF THE INVENTION

The technical domain of the invention is the treatment of fungal diseases, in particular fungal diseases due to Mucorales order.

Mucormycosis are life-threatening invasive fungal diseases due to species belonging to Mucorales order. They are difficult-to-treat infections in immunocompromised patients, being resistant to most antifungal drugs. Actual treatments are based, whenever possible, on surgery, control of underlying diseases, and aggressive antifungal therapy. Currently, there are only three active antifungal agents against Mucorales: Amphotericin B formulated as liposomes (L-AmB), recommended for first-line therapy, posaconazole (PSC) and isavuconazole (ISC), as alternative options. Despite these treatments, mortality remains inacceptable, varying from 30 to 90% depending on the disease forms.

Consequently, there is a need to provide new treatments for patient infected with fungi of the Mucorales order.

Combinations of antifungals agents have been tested in vitro and in vivo to improve their efficacy against Mucorales. Unfortunately, no combination of anti-fungal agents has been shown to be more effective than an antifungal alone against mucormycosis in patients. Several authors have therefore studied combinations of antifungal agents and non-antifungal molecules. Some of these combinations increased antifungals efficiency in vitro or in vivo in animal models, but also presented some issues such as improving Mucorales virulence or toxicity.

AmB, despite being a very active antifungal drug has a limited solubility is a highly cytotoxic substance. Toxicity of AmB and its bioaccumulation in organs, such as liver, lungs and kidneys, are the undesirable features that seriously affect the treated patients. With the development of liposomal formulations, among them the liposomal dispersion Ambisome®, the toxicity of AmB has been attenuated due to the small size of the liposomes that allows for a prolonged circulation and distribution into the organs. However, even with such liposomal formulations, there remains a problem of dose-dependent nephrotoxicity. Besides, such liposomal formulations are difficult and expensive to produce.

Cryptococcus neoformans is a yeast (fungus) involved in life-threatening invasive infections worldwide named cryptococcosis. C. neoformans is responsible for meningoencephalitis, mainly in immunocompromised patients, leading to approximately 200 000 deaths each year. This yeast therefore represents a major public health problem worldwide.

The combination of AmB and 5-fluorocytosine is currently the recommended first-line treatment. Two formulations of AmB are mainly used, AmB deoxycholate (AmBd) and liposomal AmB (L-AmB). L-AmB is the less toxic form and is preferred in developed countries. However, it is the much more expensive form and is consequently not widely used in developing countries. AmBd represents the cornerstone of the management of cryptococcosis in developing countries. Unfortunately, the efficacy of the treatment against Cryptococcus remains low with 35-40% of mortality both in in resource-rich and resource-poor settings. In this context, there is an urgent need for new treatments for cryptococcosis more efficient while being less expensive.

Thus, there is an ongoing need in the art to optimize formulations based on polyene antifungals such as AmB.

The inventors have developed formulations allowing to increase the antifungal efficacy of AmB against Mucorales and other fungi such as Cryptococcus, Aspergillus and Candida, while decreasing its toxicity.

### SUMMARY OF THE INVENTION

In a first object, the invention relates to a composition comprising an aqueous phase, a polyene antifungal and lipid nanoparticles which are dispersed in the aqueous phase, said lipid nanoparticles comprising an oily internal phase and an envelope, the envelope comprising a polyglycol ester of a hydroxy fatty acid, the polyene antifungal being dispersed in the aqueous phase and/or being part of the lipid nanoparticles.

The hydroxy fatty acid Is preferably a C8-C24 hydroxy fatty acid, preferably a C12-C20 hydroxy fatty acid, more preferably a C14-C18 hydroxy fatty acid.

Moreover, the polyglycol ester of a fatty acid preferably comprises from 1 to 100 ethylene oxide groups, preferably from 5 to 50 ethylene oxide groups, more preferably from 15 to 25 ethylene oxide groups, even more preferably 10 to 20 ethylene oxide groups.

The polyglycol ester of a fatty acid preferably comprises a C14-C18 hydroxy fatty acid, preferably a C18 hydroxy fatty acid group such as 12-hydroxysteraric acid, and from 10 to 20 ethylene oxide groups.

The polyglycol ester of a hydroxy fatty acid is polyethylene glycol-15-hydroxystearate. Moreover, the polyene antifungal may preferably be selected from amphotericin B, nystatin, partricin, natamycin or a mixture thereof.

The composition according to the invention may comprise between 1 g/L and 5 g/L, preferably between 1 g/L and 2 g/L of polyene antifungal and between 20 g/L and 100 g/L, preferably between 20 and 80 g/L, more preferably between 40 and 60 g/L of polyglycol ester of a hydroxy fatty acid. Such composition is preferably for parenteral administration.

The composition according to the invention may comprise between 30 mg/L and 120 mg/L, preferably between 50 mg/L and 100 mg/L of polyene antifungal and between 600 mg/L and 2400 mg/L, more preferably between 1000 and 2000 mg/L of polyglycol ester of a hydroxy fatty acid. Such composition is preferably for respiratory administration.

Moreover, the lipid nanoparticles have a size preferably comprised between 1 and 250 mm, preferably between 10 and 150 nm, preferably between 50 and 100 nm.

In an embodiment, the lipid nanoparticles of the composition according to the invention are free of antifungal polyene.

In an embodiment, the aqueous phase of the composition according to the invention is free of polyene antifungal.

Moreover, the lipid nanoparticles preferably comprise the polyglycol ester of a hydroxy fatty acid and the polyene antifungal in a weight ratio polyglycol ester of hydroxy fatty acid:polyene antifungal ranging from 5:1 to 100:1.

In an embodiment, the composition according to the invention further comprises a triazole antifungal or an antifungal subject to fungal resistance, in particular efflux pump-mediated resistance, wherein the triazole antifungal or the antifungal subject to fungal resistance is dispersed in the aqueous phase and/or is part of the lipid nanoparticles.

In an embodiment, the composition according to the invention further comprises a triazole antifungal or an antifungal subject to fungal resistance, in particular efflux pump-mediated resistance, wherein the triazole antifungal or the antifungal subject to fungal resistance is part of the lipid nanoparticles, preferably is dispersed in the oily core of the lipid nanoparticles.

In an embodiment, the triazole antifungal or the antifungal subject to fungal resistance is part of the lipid nanoparticles, preferably is solubilized in the oily internal phase of the nanoparticles.

In an embodiment, the composition according to the invention further comprises a triazole antifungal or an antifungal subject to fungal resistance, in particular to efflux pump-mediated resistance, solubilized in the oil phase of the nanoparticles, and the polyene antifungal solubilized in the aqueous phase and/or being part of the lipid nanoparticles.

In an embodiment, the triazole antifungal or the antifungal subject to fungal resistance and the polyene antifungal are part of the lipid nanoparticles.

The triazole antifungal may for example be selected from isavuconazole, itraconazole, posaconazole, voriconazole, fluconazole or a mixture thereof, preferably is isavuconazole.

The antifungal subject to fungal resistance, in particular subject to efflux pump-mediated resistance, is preferably a lipophilic antifungal, more preferably is an azole antifungal, in particular an azole antifungal that may be for example selected from a triazole antifungal and an imidazole antifungal.

In an embodiment, the composition comprises the triazole antifungal and the polyene antifungal in a weight ratio triazole antifungal:polyene antifungal ranging from 10 to 200, preferably from 10 to 150, more preferably from 10 to 100.

In a second object, the present invention also relates to lipid nanoparticles comprising an antifungal polyene, an oily internal phase and an envelope, the envelope comprising a polyglycol ester of a hydroxy fatty acid and the polyene antifungal being part of the lipid nanoparticles.

In an embodiment, the lipid nanoparticles further comprise a triazole antifungal or an antifungal subject to fungal resistance, in particular efflux pump-mediated resistance, dispersed in the oily internal phase of the lipid nanoparticles.

In a third object, the present invention relates to a method for preparing the lipid nanoparticles of the second object of the invention, comprising:
a) providing an oily phase comprising at least a fatty substance and an antifungal polyene as defined above,
b) providing an aqueous phase comprising a polyglycol ester of a hydroxy fatty acid,
c) mixing the oily phase of step a) and the aqueous phase of step b),
d) cooling the mixture obtained in step c) to obtain the lipid nanoparticles.

In the embodiments where the lipid nanoparticles comprise a triazole antifungal or an antifungal subject to fungal resistance, the oily phase provided in step a) further comprises a triazole antifungal.

In a fourth object, the invention relates to a composition according to the first object of the invention or lipid nanoparticles according to the second object of the invention for use in the prevention or treatment of an infection caused by a fungus, preferably a fungus of the Mucorales order.

### DESCRIPTION OF THE INVENTION

### Composition

A first object of the invention relates to a composition a comprising an aqueous phase, a polyene antifungal and lipid nanoparticles which are dispersed in the aqueous phase, said lipid nanoparticles comprising an oily internal phase and an envelope, the envelope comprising a polyglycol ester of a hydroxy fatty acid, the polyene antifungal being dispersed in the aqueous phase and/or being part of the lipid nanoparticles.

The inventors have discovered that, surprisingly, the combination of an antifungal polyene and of a polyglycol ester of a hydroxy fatty acid under the form of lipid nanoparticles allows to obtain a composition having a very low toxicity while preserving or even potentiating the efficacy of the antifungal polyene, in particular against Mucorales, whether the polyene antifungal is dispersed in the aqueous phase and/or is part of the lipid nanoparticles. The inventors have further shown that this synergy can be observed not only in Mucorales, but also in Aspergillus and Cryptococcus. Interestingly, they have further shown that the combination of an antifungal polyene, a triazole antifungal and polyglycol ester of a hydroxy fatty acid under the form of lipid nanoparticles significantly potentiates the effect of the antifungal polyene.

### Polyglycol Ester of a Hydroxy Fatty Acid

The term “polyglycol ester of a hydroxy fatty acid”, as used in the present invention, refers to a mixture comprising at least an ester of a hydroxy fatty acid obtained by the ethoxylation of a hydroxy fatty acid. The terms “polyglycol” and “poly (ethylene glycol)”, “polyethylene glycol” (PEG) or “polyethylene oxide (PEO)” or “macrogol” or “carbovax” or “polyoxyethylene” (POE) are interchangeable. The said mixture may comprise from about 60% to about 75% of esters, preferably mono- and diesters, of the hydroxy fatty acid and from about 25% to 40% of free polyethylene glycol, in wt % relative to the weight of the mixture.

The phrase “between . . . and . . . ” as used in the present invention, should be understood to include the terminals of the recited range.

Advantageously, the polyglycol ester of a hydroxy fatty acid has a Hydrophilic-Lipophilic Balance (HLB) determined by the Griffin's method comprised between 14 and 16.

The term “hydroxylated fatty acid”, as used in the present invention, refers to a fatty acid having at least one hydroxyl group attached to the alkyl chain of the fatty acid. Preferably, the fatty acid has a unique hydroxyl group attached to the alkyl chain of the fatty acid. Advantageously, the hydroxy fatty acid is a C8-C24 hydroxy fatty acid, preferably a C12-C20 hydroxy fatty acid, more preferably a C14-C18 hydroxy fatty acid. More preferably, the hydroxy fatty acid is hydroxystearic acid. More advantageously, the hydroxy fatty acid is 12-hydroxysteraric acid.

Advantageously, the polyglycol ester of a fatty acid comprises from 1 to 100 ethylene oxide groups, preferably from 5 to 50 ethylene oxide groups, more preferably from 15 to 25 ethylene oxide groups, even more preferably from 10 to 20 ethylene oxide groups. More advantageously, the polyglycol ester of a fatty acid comprises 15 ethylene oxide groups.

In an embodiment, the polyglycol ester of a fatty acid comprises a C14-C18 hydroxy fatty acid, preferably a C18 hydroxy fatty acid group such as 12-hydroxysteraric acid, and from 10 to 20 ethylene oxide groups.

In an embodiment, the polyglycol ester of a hydroxy fatty acid is polyethylene glycol-15-hydroxystearate.

In the present invention, the term “polyethylene glycol-15-hydroxystearate” refers to a mixture comprising at least an ester of 12-hydroxystearic acid obtained by the ethoxylation of 12-hydroxystearic acid. Preferably, the polyethylene glycol-15-hydroxystearate comprises at least the ester compound of formula:

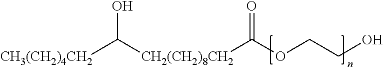

wherein n=15. Polyethylene glycol-15-hydroxystearate is also known as 12-hydroxyoctadecanoic acid polymer with a-hydro-w-hydroxypoly (oxy-1,2-ethanediyl); 12-hydroxystearic acid polyethylene glycol copolymer; macrogol 15 hydroxystearate; and polyethylene glycol 660 12-hydroxystearate. In some embodiments, the Polyethylene glycol-15-hydroxystearate is Solutol® HS 15 (CAS number 70142-34-6), also commercialized under the commercial name Kolliphor® HS 15 (BASF AG, Germany). In an embodiment, polyethylene glycol-15-hydroxystearate is a mixture comprising from about 60% to about 75% of polyglycol esters of 12-hydroxystearic acid and from about 60% to about 75% of free polyethylene glycol, in wt % relative to the weight of the mixture.

### Antifungal Polyene

The term “antifungal polyene”, as used in the present invention, refers to antifungals characterized by an almost flat macrolactone ring usually comprising eight or more atoms (usually termed polyene macrolide) with a series of conjugated double bonds. Depending on the number of conjugated double bonds, such polyenes can be classified into trienes, tetraenes, pentaenes, hexaenes, heptaenes, etc. Antifungal polyenes usually comprises a hydrophilic “head” having a mycosamine group. Such antifungal polyenes are also usually termed “antifungal macrolides”.

Advantageously, the antifungal polyene may be selected from amphotericin B (AmB), nystatin, natamycin (also known as pimaricin), partricin, or a mixture thereof. Preferably, the antifungal is amphotericin B.

The composition according to the invention may contain high concentrations of antifungal polyene without being cytotoxic. This makes the composition according to the invention particularly suitable not only for parenteral administration but also for respiratory administration, in particular for intrapulmonary respiratory administration as detailed below.

Advantageously, the composition according to the invention comprises between 1 g/L and 5 g/L, preferably between 1 g/L and 2 g/L of polyene antifungal and between 20 g/L and 100 g/L, preferably between 20 and 80 g/L, more preferably between 40 and 60 g/L of polyglycol ester of a hydroxy fatty acid. Such composition is preferably for parenteral administration and more preferably for intravenous administration as detailed below.

Advantageously, the composition according to the invention comprises between 30 mg/L and 120 mg/L, preferably between 50 mg/L and 100 mg/L of polyene antifungal and between 600 mg/L and 2400 mg/L, more preferably between 1000 and 2000 mg/L of polyglycol ester of a hydroxy fatty acid. Such composition is preferably for respiratory administration and more preferably respiratory pulmonary administration as detailed below.

Advantageously, the composition according to the invention comprise the polyglycol ester of a hydroxy fatty acid and the polyene antifungal in a weight ratio polyglycol ester of hydroxy fatty acid:polyene antifungal ranging from 5:1 to 100:1, preferably from 10:1 to 100:1, preferably from 10:1 to 90:1, preferably from 10:1 to 50:1, preferably from 10:1 to 40:1, preferably from 10:1 to 30:1, preferably from 10:1 to 25:1. Preferably, the weight ratio polyglycol ester of hydroxy fatty acid:polyene antifungal ranges from 15:1 to 25:1.

As detailed below, the composition according to the invention may comprise the antifungal polyene dispersed in the aqueous phase and/or as part of the lipid nanoparticles.

### Antifungal Triazole

In an embodiment, the composition according to the invention further comprises a triazole antifungal.

The term “antifungal triazole” as used in the present invention, refers to antifungals characterized in that they comprise a triazole fraction i.e. a five-membered ring of two carbon atoms and three nitrogen atoms, and derivatives thereof.

The triazole antifungal may for example be selected from isavuconazole, itraconazole, posaconazole, voriconazole, fluconazole or a mixture thereof. Preferably, the triazole antifungal is isavuconazole.

In an embodiment, the composition comprises the triazole antifungal and the polyene antifungal in a weight ratio triazole antifungal:polyene antifungal ranging from 10 to 200, preferably from 10 to 150, more preferably from 10 to 100.

In an embodiment, the composition according to the invention further comprises a triazole antifungal, wherein the triazole antifungal is dispersed in the aqueous phase and/or is part of the lipid nanoparticles.

In the present invention, “dispersed” may preferably mean “solubilized”.

In an embodiment, the triazole antifungal is part of the lipid nanoparticles, preferably is dispersed in the oily internal phase of the nanoparticles.

In an embodiment, the composition according to the invention comprises a triazole antifungal dispersed in the oily internal phase of the nanoparticles, and a polyene antifungal dispersed in the aqueous phase and/or being part of the lipid nanoparticles.

In an embodiment, the triazole antifungal and the polyene antifungal are dispersed in the aqueous phase.

In an embodiment, the triazole antifungal and the polyene antifungal are part of the lipid nanoparticles, the triazole antifungal preferably being dispersed in the oily internal phase of the lipid nanoparticles, the polyene antifungal preferably being dispersed in the oily core of the lipid nanoparticles and/or a constituent of the envelope.

As detailed in the experimental part below, the inventors have shown that isavuconazole greatly increases the activity of Amphotericin B when combined with a polyglycol ester of a hydroxy fatty acid.

The inventors have shown that the combination of a polyene antifungal, namely AmB and of a triazole antifungal, namely isavuconazole (ISA) in a polyglycol ester of a hydroxy fatty acid-based nanoparticle, namely PEG H15S-based nanoparticle, has a very high antifungal efficacy compared to a solution of AmB, a solution of AmB and ISA, and PEG H15S-nanoparticles comprising AmB.

The inventors believe that once incorporated into the nanoparticles of the invention, triazole antifungals, which cannot be administered by nebulization or with very low efficiency at present, would become usable by nebulized route. Without wishing to be bound by any particular theory, the inventors believe that, because triazoles are very lipophilic, they are absorbed very quickly into the bloodstream when administered intrapulmonarily as an aerosol, and therefore not have time to act on the fungus in the lung. The inventors believe that in contrast, if triazoles are administered as aerosolized nanoparticles as according to the invention, the absorption of the triazoles from the lungs should be slowed down, which would increase their pulmonary residence time, thus giving them more time to exert antifungal action in the lungs.

According to the inventors, the interest of incorporating the combination of a polyene antifungal such as AmB and a triazole antifungal such as isavuconazole in nanoparticles according to the invention would thus be twofold, that of decreasing the MIC of the said antifungals in a surprising manner, while at the same time rendering it possible to administer the triazole by nebulization with highly increased pulmonary residence time.

In an embodiment, the composition of the invention comprises a polyene antifungal and a triazole antifungal, wherein the polyene antifungal is preferably AmB and the triazole antifungal is preferably isavuconazole.

### Antifungal Subject to Resistance

The inventors have further shown that the combination of a polyene antifungal, namely AmB in a polyglycol ester of a hydroxy fatty acid-based nanoparticle, namely PEG H15S-based nanoparticle, penetrate Cryptococcus cells much more effectively than nanoparticles not loaded with Amb. Indeed, as shown in the experimental part of the present application, lipid nanoparticles according to the invention comprising AmB as polyene antifungal penetrated Cryptococcus cells 16 times more efficiently than PEG H15S-based nanoparticle not comprising polyene antifungal.

Without being bound by a theory, the inventors believe that these data suggest that the lipid nanoparticles according to the invention, when comprising a polyene antifungal in combination with an antifungal subject to fungal resistance such as isavuconazole, could be of help in enhancing the intracellular concentration of the antifungal subject to fungal resistance into fungal cells. Indeed, some antifungals such as azoles (including isavuconazole) may be substrates of fungal membrane efflux pumps, which decreases their efficiency by decreasing their intracellular concentration, resulting in antifungal resistance, e.g. azole resistance. The use of lipid nanoparticles according to the invention could allow to bypass the effect of these pumps, resulting in higher intracellular concentration of antifungals subject to fungal resistance. The lipid nanoparticles according to the invention could thus be of use in overcoming fungal resistance, in particular efflux pump-mediated fungal resistance.

In an embodiment, the lipid nanoparticles according to the invention comprise an antifungal that is subject to fungal resistance, in particular to efflux pump-mediated resistance.

The expression “antifungal subject to efflux pump-mediated resistance” or “antifungal subject to EPMR”, as used in the present invention, refers to an antifungal that may be difficult or impossible to accumulate intracellularly in some fungal cells up to toxic levels due to the activity of fungal membrane-associated transporters acting as efflux pumps, rendering the antifungal treatment less effective or ineffective. Indeed, it is known that some fungal membrane-associated transporters, such as primary active transporters pertaining to the superfamily of ATP-binding cassette (ABC) proteins, or secondary active transporters belonging to major facilitator superfamily (MFS), can recognize some antifungals as substrates, resulting in an antifungal efflux from the fungal cells. In some resistant fungal strains, the fungal efflux pumps ensures that the antifungal is not accumulated to lethal levels.

In an embodiment, the lipid nanoparticles according to the invention comprise an antifungal polyene, preferably AmB, and an antifungal subject to resistance, in particular subject to EPMR, preferably an azole antifungal. The polyene antifungal is preferably dispersed in the oily core of the lipid nanoparticles and/or is a constituent of the envelope, preferably at least a part of the polyene antifungal is part of the enveloppe. The antifungal subject to fungal resistance is preferably a lipophilic antifungal dispersed in the oily core of the lipid nanoparticles.

In an embodiment, the antifungal subject to fungal resistance, in particular subject to EPMR, is a lipophilic antifungal, preferably is an azole antifungal.

The term “azole antifungal” as used in the present invention, refers to antifungals characterized in that they comprise five-membered nitrogen containing heterocyclic ring systems containing other non-carbon atom(s) such as sulfur or oxygen, and derivatives thereof. The azole group of antifungal drugs is usually divided into two subgroups: the triazole antifungals as defined above and the imidazole antifungals. Examples of imidazole antifungals are clotrimazole, econazole, miconazole, ketoconazole.

In an embodiment, the lipid nanoparticles according to the invention comprise an antifungal polyene, preferably AmB, and an azole antifungal. The azole antifungal may be a triazole antifungal, preferably isavuconazole, or an imidazole antifungal.

### Lipid Nanoparticles (LNPs)

The term “lipid nanoparticles”, as used in the present invention, relates to a nanometer-size lipid particle of vesicular type comprising an oily internal phase (or an oily core) and an envelope (or a shell) comprising a polyglycol ester of a hydroxy fatty acid.

The size of the lipid nanoparticles is preferably between 1 and 250 mm, preferably between 10 and 150 nm, preferably between 50 and 100 nm.

The polydispersity index (PDI) of the lipid nanoparticles according to the invention is preferably lower than 0.4, more preferably lower than 0.2.

The sizes given above correspond to the Z average, i.e. the mean value of the intensity-weighted size distribution of the lipid nanoparticles as determined by dynamic light scattering at 25° C. using a Zetasizer Nano ZS apparatus from Malvern Instrument (Malvern Panalytical).

As used herein, PDI is a number calculated from a two-parameter fit to the correlation data (the cumulants analysis). This index is dimensionless and scaled such that values smaller than 0.05 are mainly seen with highly monodisperse standards. PDI values bigger than 0.7 indicate that the sample has a very broad particle size distribution and is probably not suitable to be analyzed by the dynamic light scattering (DLS) technique. Different size distribution algorithms work with data that fall between these two extreme values of PDI (i.e., 0.05-0.7). In the present invention, the size and PDI parameters are determined as defined in the ISO standard documents 13321:1996 E and ISO 22412:2008.

The oily core, which is liquid or semi-liquid at room temperature, contains a biocompatible and biodegradable lipophilic compound with low water miscibility preferably selected from vegetable oils such as soybean oil, corn oil, olive oil, safflower oil, triglycerides; tocopherols such as alpha-tocopherol, beta-tocopherol, gamma-tocopherol, delta-tocopherol, alpha-tocotrienol, beta-tocotrienol, gamma-tocotrienol, delta-tocotrienol; fish oils: sterols; vitamin D such as cholecalciferol and ergocalciferol; or mixtures thereof.

The terms “biocompatible and biodegradable”, as used in the present invention, refer to a compound that may be absorbed and eliminated by the body, preferably with little accumulation in the tissues.

The triglycerides can comprise one or more triglycerides, in particular one or more triglycerides of at least one fatty acid, preferably a C6-C12 fatty acid, more preferably a C6-C10 fatty acid. A commercially available triglyceride composition that may be used is Labrafac lipophile WL 1349, available from Gattefosse Corporation.

In addition to the nonionic surfactant, the envelope may comprise other excipients to stabilize the shell, particularly selected from an amphoteric surfactant such as a phospholipid or an anionic surfactant such as a fatty acid.

Advantageously, the amphoteric surfactant is an amphiphilic lipid chosen from the phospholipid family, in particular from phosphatidylcholines or lecithins. The amphiphilic lipid used in the present invention is preferably solid at ambient temperature, which promotes the formation of a semi-solid interface around the liquid or semi-liquid core.

The amphiphilic lipid is preferably chosen from the group of lecithins, more preferably lecithins exhibiting a transition temperature of greater than 35° C., such as dipalmitoylphosphatidylcholine (DPPC), distearoylphosphatidylcholine (DSPC), dibehenyl phosphatidylcholine (DBPC), palmitoyl-stearoyl phosphatidylcholine (PSPC), palmitoyl-behenyl phosphatidylcholine (PSPC), stearoyl-behenyl phosphatidylcholine (SBPC).

The lipid nanoparticles preferably contain a lecithin which is hydrogenated. More particularly, the hydrogenated lecithin used according to the invention has a high percentage of saturated phosphatidylcholine. The term “high percentage” is understood to mean an amount of greater than 85% of hydrogenated (or saturated) phosphatidylcholine relative to the total weight of lecithin. A commercially available lecithin composition that may be used is Phospholipon® 90 H for which the hydrogenated phosphatidylcholine content is greater than 90%, the transition temperature of which is approximately 54° C.

Advantageously, the lipid nanoparticles comprise the amphiphilic lipid compound and the polyglycol ester of a hydroxy fatty acid in a weight ratio amphiphilic lipid:polyglycol ester of a hydroxy fatty acid ranging between 10:2 to 25:1.

Advantageously, the lipid nanoparticles, particularly the envelope of the lipid nanoparticles according to the invention, further comprise a sterol.

The sterol may be selected from zoosterols such as cholesterol, phytosterols such as campesterol, sterol derivatives such as vitamin D, or mixtures thereof.

Advantageously, the lipid nanoparticles comprise a sterol in an amount of between 0% and 15% by weight, preferably between 5% and 10% by weight relative to the total weight of the composition according to the invention.

In the composition according to the invention, the polyene antifungal is dispersed in the aqueous phase and/or is part of the lipid nanoparticles.

The expression “part of the lipid nanoparticles”, referring to the antifungal polyene, means that the antifungal polyene is dispersed in the oily core of the lipid nanoparticles and/or is a constituent of the envelope. In an embodiment, the antifungal polyene is embedded into the envelope and/or adsorbed onto the outer surface of the envelope.

The expression “part of the lipid nanoparticles”, referring to the antifungal triazole, means that the antifungal polyene is dispersed in the oily core (i.e. the oily internal phase) of the lipid nanoparticles and/or is a constituent of the envelope. In a preferred embodiment, the antifungal triazole is dispersed in the oily internal phase of the lipid nanoparticles. In another embodiment, the antifungal triazole is embedded into the envelope and/or adsorbed onto the outer surface of the envelope.

As mentioned above, the term “dispersed” preferably means “solubilized”.

In a particular embodiment, the composition according to the invention comprises the antifungal polyene dispersed in the aqueous phase of the composition.

In this embodiment, the lipid nanoparticles are preferably free of antifungal polyene. An advantage of this embodiment is that it is cheaper, less complex to prepare and potentially more stable than a composition having lipid nanoparticles comprising antifungal polyene.

The inventors have shown that, surprisingly, the toxicity of AmB dispersed in aqueous phase was significantly decreased in presence of PEG15HS incorporated in lipid nanoparticles, when compared to a composition comprising both AmB and PEG15HS in solution.

In another embodiment, the antifungal polyene is part of the lipid nanoparticles. In this embodiment, the aqueous phase is free of antifungal polyene. Preferably, the antifungal polyene is AmB.

An advantage of this embodiment is that it ensures that the weight ratio polyglycol ester of hydroxy fatty acid:polyene antifungal is maintained up to the side of action of the polyene antifungal. Without wanting to be bound by a particular theory, the inventors believe that there is a risk that the polyglycol ester of a hydroxy fatty acid and the polyene antifungal do not arrive at the site of action in an optimal ratio when the antifungal polyene is in the aqueous phase.

In an embodiment, the lipid nanoparticles comprise a polyene antifungal and a triazole antifungal, wherein the polyene antifungal is preferably AmB and the triazole antifungal is preferably isavuconazole.

An advantage of this embodiment is that, as shown in the experimental part below, the combination of a polyene antifungal such as AmB and of a triazole antifungal such as isavuconazole (ISA) in a Polyglycol ester of a hydroxy fatty acid-based nanoparticle, such as a PEG H15S-based nanoparticle, has a very high antifungal efficacy compared to a solution of AmB, a solution of AmB and ISA, and PEG H15S-nanoparticles comprising AmB. Thus, an advantage of that embodiment is that the combination of a polyene antifungal and a triazole antifungal in the lipid nanoparticles according to the invention decreases the MIC of both the polyene antifungal and of the triazole antifungal.

A further advantage of the nanoparticles of this embodiment is that the incorporation of the triazole antifungal into the lipid nanoparticles according to the invention would slow down the absorption of the triazole antifungal by the lungs, thereby increasing its residence time and thus its in vivo antifungal efficacy. This would among other making it interesting to administer such lipid nanoparticles by nebulization.

In an embodiment, the lipid nanoparticles comprise a polyene antifungal and an antifungal subject to fungal resistance, wherein the polyene antifungal is preferably AmB and the antifungal subject to fungal resistance is preferably an azole antifungal such as a triazole antifungal or an imidazole antifungal.

An advantage of this embodiment is that such lipid nanoparticles could be of help in overcoming fungal EPMR, as detailed above.

In a second object, the invention relates to lipid nanoparticles as defined in the first object according to the invention, wherein the lipid nanoparticles comprise the antifungal as a part of the lipid nanoparticles.

Advantageously, these lipid nanoparticles comprise the polyglycol ester of a hydroxy fatty acid and the polyene antifungal in a weight ratio polyglycol ester of hydroxy fatty acid:polyene antifungal ranging from 400:1 to 50:1, preferably from 300:1 to 50:1, more preferably from 200:1 to 50:1, even more preferably from 125:1 to 75:1.

Advantageously, these lipid nanoparticles comprise between 5% and 60% by weight, preferably between 5% and 50% by weight and more preferably between 30% and 40% by weight of polyglycol ester of a hydroxy fatty acid, in % by weight relative to the total weight of the lipid nanoparticles.

Advantageously, these lipid nanoparticles comprise between 0.01% and 10% by weight, preferably between 0.1% and 5%, and more preferably between 0.5 and 2% by weight of polyene antifungal, in % by weight relative to the total weight of the lipid nanoparticles.

### Method for Preparing the Lipid Nanoparticles According to the Invention

In a third object, the process relates to a method for preparing the lipid nanoparticles according to the second object of the invention, comprising the following steps:
a) providing an oily phase comprising at least a fatty substance and an antifungal polyene as defined above,
b) providing an aqueous phase comprising a polyglycol ester of a hydroxy fatty acid,
c) mixing the oily phase of step a) and the aqueous phase of step b),
d) cooling the mixture obtained in step c) to obtain the lipid nanoparticles.

When the lipid nanoparticles comprise a triazole antifungal, the oily phase provided in step a) further comprises a triazole antifungal.

Preferably, the step c) is carried out applying temperature cycles between 90° C.-60° C. with steps of 4° C./min. Preferably, 2, 3, 4 or 5 temperature cycles are carried out.

Preferably, the step d) of cooling the mixture obtained in step c) is carried out by cooling down the mixture to a temperature comprises below 60° C., preferably to a temperature comprised superior to 55° C. and inferior to 60° C., even more preferably to a temperature of 58° C. to obtain the lipid nanoparticles.

The mixing step c) may be carried out via techniques that are known to those skilled in the art, for example by high pressure homogenization, high-speed stirring, ultra-sonication, phase inversion temperature (PIT), or solvent injection.

In an embodiment, the lipid nanoparticles of the invention are in the form of a suspension or in the form of a dry powder.

In the latter, lipid nanoparticles of the invention under the form of dry powders may be obtained by spray-drying or freeze-drying aqueous dispersions of the lipid nanoparticles, said aqueous dispersions preferably comprising stabilizing agents. Such stabilizing agents are preferably selected from sugars such as lactose, trehalose, and raffinose; cyclodextrins; biopolymers such as starch and dextrins; synthetic polymers having a molecular weight higher than 5000 g/mol but lower than 25 000 g/mol such as polyvinyl alcohol, polyvinylpyrrolidone (PVP), and linear or branched polyethylene glycol, or mixtures thereof.

### Method of Treatment

In a fourth object, the invention relates to the composition according to the first object invention or the lipid nanoparticles according to the second object invention for the prevention or treatment of an infection caused by a fungus.

According to one embodiment, the infection is an infection caused by at least one fungus of the Mucorales order, preferably at least one fungus of the genus selected in the group comprising but not limited to the Rhizopus (e.g. R. arrhizus spp., R. microsporus spp.), Lichtheimia (e.g. L. ramosa spp., L. corymbifera spp.), Rhizomucor (e.g. R. pusillus spp.), Mucor (e.g. M. circinelloides spp.), Cunninghamella (e.g. C. bertholletiae), Saksenaea (e.g. S. vasiformis) and Apophysomyces (e.g. A. elegans).

According to another embodiment, the infection is an infection caused by at least one fungus of the genus selected in the group comprising but not limited to Aspergillus (e.g. A. clavatus, A. flavus, A. fumigatus, A. felis, A. nidulans, A. niger, A. terreus, A. lentulus or A. versicolor), Fusarium (e.g. F. oxysporum, F. solani, F. verticillioides, F. chlamydosporum, F. dime rum, F. fujikuroi, or F. incarnatum), Scedosporium (e.g. S. apiospermum, S. auraticum, S. boydii or S. prolificans), Lomentospora (e.g. Lomentospora prolificans), Blastomyces (e.g. B. capitatus, B. dermatitidis and B. brasiliensis), Coccidioides (e.g. C. immitis, C. posadasii), Histoplasma (e.g. Histoplasma capsulatum), Candida (e.g. C. albicans, C. parapsilosis, C. tropicalis, C. krusei, C. dubliniensis, C. famata, C. glabrata, C. auris) and Cryptococcus (e.g. C. gattii, C. albidus, C. laurentii or C. neoformans).

According to an embodiment, the infection is caused by one fungus of the genus Aspergillus, in particular Aspergillus fumigatus.

According to an embodiment, the infection is caused by one fungus of the genus Cryptococcus, in particular is caused by Cryptococcus gattii or Cryptococcus neoformans, preferably is caused by Cryptococcus neoformans.

According to an embodiment, the infection is caused by a fungus showing an antifungal resistance, preferably showing an efflux pump-mediated resistance, more preferably showing an azole resistance, wherein the fungus may in particular be of the Mucorales order or of a genus selected in the group comprising but not limited to the Rhizopus (e.g. R. arrhizus spp., R. microsporus spp.), Lichtheimia (e.g. L. ramosa spp., L. corymbifera spp.), Rhizomucor (e.g. R. pusillus spp.), Mucor (e.g. M. circinelloides spp.), Cunninghamella (e.g. C. bertholletiae), Saksenaea (e.g. S. vasiformis) and Apophysomyces (e.g. A. elegans), Aspergillus (e.g. A. clavatus, A. flavus, A. fumigatus, A. felis, A. nidulans, A. niger, A. terreus, A. lentulus or A. versicolor), Fusarium (e.g. F. oxysporum, F. solani, F. verticillioides, F. chlamydosporum, F. dime rum, F. fujikuroi, or F. incarnatum), Scedosporium (e.g. S. apiospermum, S. auraticum, S. boydii or S. prolificans), Lomentospora (e.g. Lomentospora prolificans), Blastomyces (e.g. B. capitatus, B. dermatitidis and B. brasiliensis), Coccidioides (e.g. C. immitis, C. posadasii), Histoplasma (e.g. Histoplasma capsulatum), Candida (e.g. C. albicans, C. parapsilosis, C. tropicalis, C. krusei, C. dubliniensis, C. famata, C. glabrata) and Cryptococcus (e.g. C. gattii, C. albidus, C. laurentii or C. neoformans).

According to an embodiment, the infection is caused by a fungus showing an antifungal resistance, preferably showing an efflux pump-mediated resistance, more preferably showing an azole resistance, wherein the fungus is of the Aspergillus, Candida, or Cryptococcus genus. The present invention provides according to yet another aspect, methods for preventing or treating diseases and disorders caused by fungus species, comprising administering to the subject in need thereof said composition or lipid nanoparticles.

According to some embodiments the said composition or lipid nanoparticles are administered at an advanced stage of a fungus infection, preferably an infection by a fungus of the Mucorales order.

The present invention also relates to the use of said composition or lipid nanoparticles as described herein for the manufacture of a medicament for preventing or treating diseases and disorders caused by fungus species.

### Mucormycosis

As used herein, the term “mucormycosis” refers to an infection caused by a fungus of the Mucorales order.

According to some embodiments, the said composition or lipid nanoparticles are useful for the treatment of diseases caused by a fungus of the Mucorales order, advantageously selected from the group consisting of rhinocerebral (sinus and brain) mucormycosis, pulmonary (lung) mucormycosis, gastrointestinal mucormycosis, cutaneous (skin) mucormycosis and disseminated mucormycosis. According to currently preferred embodiments, the methods of the invention are useful for the treatment of pulmonary mucormycosis and disseminated mucormycosis.

In an embodiment, the mucormycosis is caused by a fungus of the Mucorales order showing an antifungal resistance, preferably showing an efflux pump-mediated resistance, more preferably showing an azole resistance.

### Aspergillosis

As used herein, the term “aspergillosis” refers to an infection caused by a fungus of the Aspergillus genus.

According to an embodiment, the said composition or lipid nanoparticles are useful for the treatment of diseases caused by a fungus of the Aspergillus genus, advantageously selected from the group consisting of invasive aspergillosis, chronic pulmonary aspergillosis, allergic bronchopulmonary aspergillosis, Aspergillus bronchitis.

In an embodiment, the aspergillosis is caused by a fungus of the Aspergillus genus showing an antifungal resistance, preferably showing an efflux pump-mediated resistance, more preferably showing an azole resistance.

### Cryptococcosis

As used herein, the term “cryptococcosis” refers to an infection caused by a fungus of the Cryptococcus genus, most often caused by Cryptococcus neoformans.

According to some embodiments, the said composition or lipid nanoparticles are useful for the treatment of diseases caused by a fungus of the genus Cryptococcus, and advantageously selected from cryptococcal Meningoencephalitis, pulmonary cryptococcosis, cutaneous cryptococcosis. In an embodiment, the said composition or lipid nanoparticles are useful for the treatment of Cryptococcus neoformans Meningoencephalitis.

In an embodiment, the cryptococcosis is caused by a fungus of the Cryptococcus genus showing an antifungal resistance, preferably showing an efflux pump-mediated resistance, more preferably showing an azole resistance.

### Administration Mode

The composition or lipid nanoparticles of the invention can be administered by any convenient route of administration. Except otherwise stated, the routes of administrations are defined as detailed in the standard FDA C-DRG-00301 dated Nov. 14, 2017.

In an embodiment, the composition or lipid nanoparticles of the invention are for enteral administration (i.e. administration directly into the intestines), oral administration (i.e. administration to or by way of the mouth), sublingual administration (i.e. administration beneath the tongue), buccal administration (i.e. administration directed toward the cheek, generally from within the mouth), or rectal administration (ie. Administration to the rectum).

In an embodiment, the composition or lipid nanoparticles of the invention are for parenteral administration (i.e. administration by injection, infusion, or implantation). As used herein parenteral administration includes intravenous administration, intramuscular administration, subcutaneous administration.

The composition or lipid nanoparticles of the invention for parenteral administration are preferably in the form of an injectable suspension, in particular for intravenous administration.

The composition or lipid nanoparticles of the invention for parenteral administration, preferably intravenous administration, are preferably administered in the form of an injectable suspension allowing the maximum concentrations of antifungal polyene achieved in the plasma to be comprised between 1 and 15 mg/L, preferably between 1 and 10 mg/L, preferably between 1 and 5 mg/L.

In an embodiment, the composition or lipid particles are for respiratory administration (i.e. administration within the respiratory tract by inhaling e.g. orally, herein also termed inhalation administration), preferably for intrapulmonary respiratory administration.

As used herein, “intrapulmonary respiratory administration”, refers to a respiratory administration allowing deliver the composition or the lipid nanoparticles to the lungs or its bronchi, where it concentrates at the alveolar epithelium. Pulmonary administration comes a complement to a parenteral (preferably intravenous) administration.

The composition or lipid nanoparticles of the invention for respiratory administration, preferably respiratory pulmonary administration, can be administered as an aerosol of a powder, of an aqueous solution or of an aqueous suspension based on the composition or the lipid nanoparticles of the invention.

The composition or lipid nanoparticles of the invention for respiratory administration, preferably respiratory pulmonary administration, are preferably administered in the form of an aerosol allowing the maximum concentrations of antifungal polyene reached in the pulmonary epithelial lining fluid, as measured by bronchoalveolar lavage, to be comprised between 15 mg/L and 60 mg/L, preferably between 15 and 60 mg/L, most preferably between 30 and 60 mg/L.

The term “aerosol”, as used in the present invention, refers to a dispersion of solid particles or liquid droplets in a gas adapted for targeting the lower airway passages, and preferably the lungs. The composition or the lipid nanoparticles of the invention for respiratory administration preferably comprise a discontinuous phase of lipid nanoparticles of the invention or of liquid droplets of the composition of the invention dispersed in a continuous gas phase.

The composition or lipid nanoparticles of the invention for respiratory administration, preferably respiratory intrapulmonary administration can be administered using a nebulizer or a dry powder inhaler.

A nebulizer is defined as a device capable of aerosolizing a liquid material (solution or dispersion) in the form of inhalable liquid droplets. The nebulizer allows the administration of said composition by means of a mask or a tip disposed on the mouth and/or the nose of the subject. In a particular embodiment of the invention, an aqueous suspension of the lipid nanoparticles of the invention are administered with the aid of a nebulizer, that may for example be a jet nebulizer or a mesh nebulizer.

The composition or lipid nanoparticles of the invention are useful for treating a fungus infection, in particular an infection caused by a fungus of the Mucorales order, of the Aspergillus genus or of the Cryptococcus genus that is, the active ingredients are contained in an amount to achieve their intended purpose. At this scope, the said composition or nanoparticles should be administered in an effective amount.

The term “effective amount” refers to an amount of the compound sufficient to produce the desired therapeutic result. In particular the said composition of lipid nanoparticles of the invention are administered in amounts that are sufficient to display a desired effect.

Optimal dosages of said composition or lipid nanoparticles of the invention to be administered may be readily determined by those skilled in the art and will vary with the composition or lipid nanoparticles used, the strength of the preparation, the mode of administration, and the severity of the infection to be treated. In addition, factors associated with the particular patient being treated, including patient age, weight, diet and time of administration, will result in the need to adjust dosages and interval. The frequency and/or dose relative to the simultaneous or separate administrations can be adapted by one of ordinary skill in the art, in function of the patient, the pathology, the form of administration, etc.

The invention will be understood more clearly by means of the examples which follow, which are illustrative and nonlimiting.

### BRIEF DESCRIPTION OF THE DRAWINGS

FIG. 1: MIC of amphotericin B (AmB) against Lichtheimia ramosa measured in the presence of AmB in solution in RPMI and PEG15HS under the form of blank lipid nanoparticles (BLNPs) dispersed in RPMI. The curves correspond to duplicates of the same assay.

FIG. 2: Hemolytic activity on human erythrocytes dispersed in PBS of AmB (1, 5, 15 mg/L) solubilized in PBS mixed with PEGHS15 (0, 10, 20, 50, 100, 200, 400, 800, 1600, 3200 mg/L) loaded in BLNPs.

FIG. 3: Hemolytic activity on human erythrocytes dispersed in RPMI of AmB (15, 30, 45, 60 mg/L) solubilized in RPMI mixed with PEGHS15 (0, 10, 20, 50, 100, 200, 400, 800, 1600, 3200, 6400 mg/L) loaded in BLNPs. A15: AmB at 15 mg/L; A30: AmB at 30 mg/L; A45: AmB at 45 mg/L; A60: AmB at 60 mg/L.

FIG. 4: Percentage of hemolysis as a function of a formulation of LNPs comprising PEG15HS and 15 mg/L AmB (LNP) or LNPs comprising PEG15HS, cholesterol and 15 mg/L AmB (LNP+CH) in PBS. AMB: control assay using 15 mg/L AmB dispersed in PBS.

FIG. 5: Comparison of AmB concentration in LNPs comprising AmB and PEG15HS (LNP-009 and LNP-014), LNPs comprising AmB and Brij® S20 (LNP-015) and LNPs comprising AmB Brij® O20 (LNP-016).

FIG. 6: Comparison of percentage of hemolytic activity of LNPs comprising AmB and PEG15HS (LNP-014), LNPs comprising AmB and Brij® S20 (LNP-015) or Brij® O20 (LNP-016). AMB: hemolytic activity of 15 mg/L of AmB in solution. Red cells and LNPs or AmB were in PBS pH=7.4 and incubated 1 hour at 37° C.

FIG. 7: Structure of compounds characteristic of A) Solutol® HS15 with n=15; B) Brij® O20 with n=20; C) Brij® S10 with n=10, or Brij® S20 if n=20; D) Brij® C10 with n=10.

FIG. 8: AmB MICs (mg/L) versus PEG15HS concentrations (mg/L) for twelve Mucorales strains. AmB and PEG15HS are dispersed in an aqueous phase. Circles represent the AmB MICs determined during one checkerboard experiment and the solid lines the individual AmB MICs predicted by the Emax model based on three checkerboard experiments.

FIG. 9: MIC of AmB against Lichtheimia ramosa measured for different concentrations of surfactants as solutions. AmB and the different surfactants are solubilized in an aqueous phase. Circles represent the AmB MICs determined during one checkerboard experiment and the solid lines the individual AmB MICs predicted by the Emax model based on three checkerboard experiments.

FIG. 10: Hemolytic activity of AmB, surfactants and combinations of AMB-surfactants. Hemolytic activity of Solutol® HS15, Brij® O20 and Brij® C10 (0, 10, 20, 50 and 100 mg/L), AmB (5 mg/L) and combinations of AmB (5 mg/L) and Solutol® HS15, Brij® O20 or Brij® C10 (0, 10, 20, 50 and 100 mg/L) was measured on human erythrocytes dispersed in RPMI (a) or in PBS (b) after 1 hour of incubation at 37° C. Hemolysis was measured at 540 nm using a plate reader. Experiments were performed in duplicate. AmB 5: AmB at 5 mg/L.

FIG. 11: Hemolytic activity of AmB, Solutol® HS15 and combinations of AMB-Solutol® HS15. Hemolytic activity of AmB (0, 1, 5, 15, 30, 45, 60 mg/L), PEG15HS (0, 10, 20, 50, 100, 200 and 400 mg/L) and combinations of AmB (0, 1, 5, 15, 30, 45, 60 mg/L) and PEG15HS (0, 10, 20, 50, 100, 200 and 400 mg/L) in solution was measured on human erythrocytes dispersed in RPMI (FIG. 11a) and in PBS (FIG. 11b) after 1 hour of incubation at 37° C. Hemolysis was measured at 540 nm using a plate reader. Experiments were performed in duplicate on two experiments. ns: non significative; *p<0.05.

FIG. 12: Fluorescence intensity ratios between the Cryptococcus cells marker (CellTracker Green CMFDA) and the lipid nanoparticles marker (DiL) 18 hours after incubation.

### EXAMPLES

In the following Examples, unless otherwise stated, the properties of the formulations were measured with the following methods:

### Minimum Inhibitory Concentrations (MIC)

MICs for each fungi were measured in the presence of different concentrations of AmB (solubilized in the LNP suspensions or loaded in the LNP) as described by the EUCAST broth microdilution antifungal susceptibility test methodology (EUCAST DEFINITIVE DOCUMENT E.Def 9.3.2 Apr. 2020. EUCAST antifungal MIC method for mould).

### Hemolytic Activity

Human blood samples from different healthy volunteers was collected in EDTA tubes and centrifuged at 3000×g for 5 minutes. Plasma was removed and the erythrocytes were washed four times with 2 ml of 0.9% NaCl by centrifugation at 3000×g for 5 minutes.

Erythrocytes were suspended in PBS pH 7.4 and their concentration was adjusted to 5·10\hat{ }8 cells/mL. Erythrocytes were then incubated with AmB solubilised as a solution or loaded in LNP for 1 hour at 37° C. under shaking, then centrifuged at 3000×g for 5 minutes. Then, 100 μL of the supernatant were transferred in a flat-bottomed transparent 96-well plate and the absorbance was measured at 540 nm. Untreated erythrocytes dispersed in PBS were used as a negative control, and erythrocytes treated by 1% (v/v) of TritonX-100 were used as a positive control. Percentage of hemolysis was calculated using the following equation: 100*(Asample−Acontrol)/(aTritonX−100−Anegative), where Asample is the absorbance measured for each experimental conditions, Anegative and aTritonX-100 are the absorbance of negative and positive controls, respectively.

### Lipid Nanoparticle Preparation

Lipid nanoparticles comprising (LNPs) or not comprising (BLNPs) AmB were prepared following the procedure described by Anton et al. (Anton, N.; Gayet, P.; Benoit, J.-P.; Saulnier, P. Nano-emulsions and nanocapsules by the PIT method: An investigation on the role of the temperature cycling on the emulsion phase inversion. Int. J. Pharm. 2007, 344, 44-52).

Briefly, the oil phase of the lipid nanoparticles made of triglycerides (Labrafac®) was mixed under magnetic stirring with water in the presence of NaCl and two surfactants (Solutol HS15® and Phospholipon®). This oil-in-water emulsion was heated above the phase-inversion temperature (PIT) (90° C.), to obtain a water-in-oil emulsion. Then, it was cooled down below the PIT at 60° C., leading back to the formation of an oil-in-water emulsion. Three of those temperature cycles were carried out, and the system was then quenched by adding 12-10 ml of cold water (4° C.).

### Example 1: Composition Comprising AmB Dispersed in the Aqueous Phase and Lipid PEG15HS-Based Nanoparticles not Comprising AmB (BLNPs) Material and Methods

Formulations comprising AmB in the form of a solute and increasing concentrations of PEG15HS incorporated in lipid nanoparticles (LNPs) were prepared and assayed. The LNPs do not comprise AmB and are referred to as “blank lipid nanoparticles” (BLNPs) in the following.

### 1.1.1 Preparation of Lipid Nanoparticles Comprising PEG15HS

BLNPs comprising increasing concentrations of PEG15HS were prepared as detailed above. The ingredients are detailed in Table 1.

TABLE
composition of BLNPs

| | Mass (mg) |
|---|---|
| Solutol ® HS15 (PEG15HS) | 850 |
| Phospholipon ® | 75 |
| Labrafac lipophile WL1349 | 1028 |

### 1.1.2. Preparation of Formulations Comprising AmB in Solution and BLNPs.

The BLNPs are then mixed with a solution containing 1, 5, 15, 30, 45, or 60 mg/ml of AmB as a solute.

AmB—BLNPs mixtures were prepared by mixing a volume of BLNPs as prepared in 1.1.1 with the same volume of two-fold concentrated solutions of AmB in RPMI or DPBS.

Liquid formulations containing AmB in dissolved form and PEG15HS incorporated in LNPs dispersed in the liquid formulation are obtained.

### 1.1.3. Strains

A clinical isolate of Lichtheimia ramosa was used. Isolate were subcultured from frozen stocks (−80° C.) on 75 cm³ flasks containing 15 ml of Sabouraud dextrose agar (Sigma-Aldrich, St. Quentin Fallavier, France) during 3 days at 37° C. Spores were collected by flooding the flask with 10 mL of sterile water. The suspensions were filtered on nylon filter (Millipore, Carrigtwohill, Ireland) with pore size of 11 μm to remove hyphal elements. The concentration of the spore suspensions was then adjusted in sterile water to obtain inocula of 2×10⁵ spores/mL.

### 1.1.3. Media

RPMI 1640 (with L-glutamine, pH indicator, no bicarbonate) (Sigma-Aldrich) supplemented with 2% (w/v) dextrose and buffered to pH 7 with 0.165 mol/L MOPS (Sigma-Aldrich) was used for MIC and hemolytic activity measurements. This medium was sterilized using 0.22 μm pore-size filter. Sterile Dulbelcc”s phosphate buffered saline (DDPBS, Dominique Dutscher, Bernolsheim, France) pH 7 was also used as a medium to measure the hemolytic activity of AmB.

### 1.2. Results

### 2.2.1. Efficacy Against Mucorales

The MIC of the formulations for Lichtheimia ramosa was assayed.

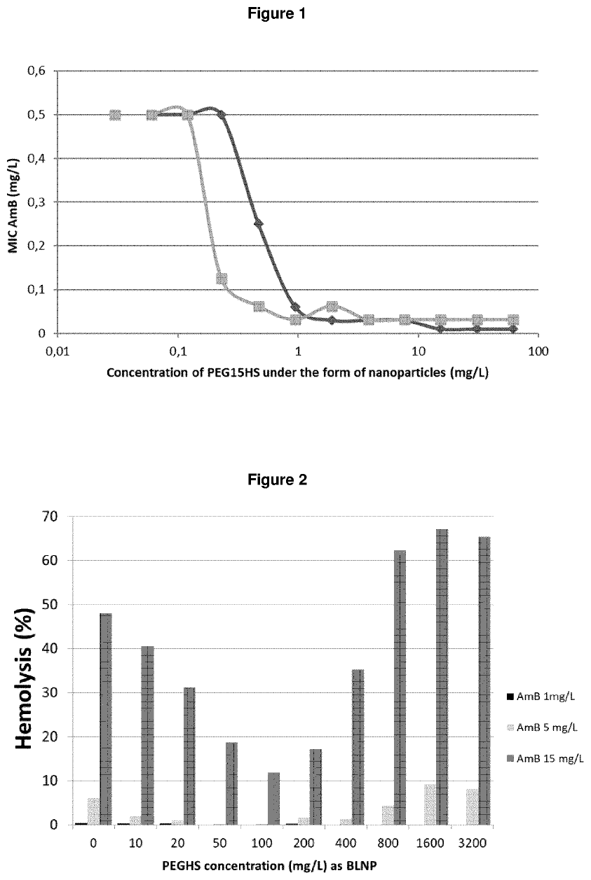

The results are shown in FIG. 1.

The results show that in the presence of 15-30 mg/L of PEG15HS incorporated in LNPs, the MIC of AmB decreases from 0.5 mg/L to 0.01 mg/L.

### 2.2.1. Hemolytic Activity

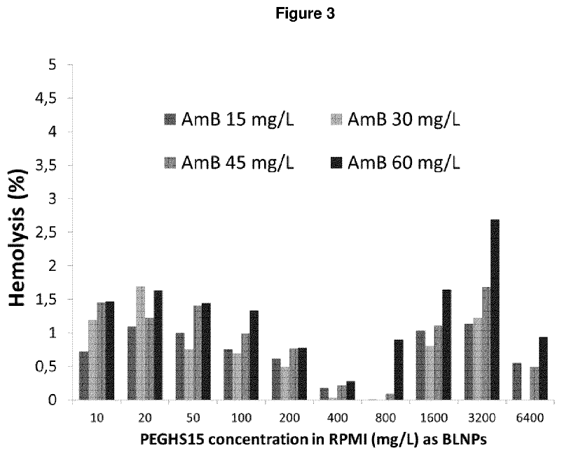

The results are shown in FIG. 3.

In PBS, 5 to 15 mg/L of AmB induce hemolysis. PEG15HS loaded in LNPs reduces this hemolytic effect of AmB up to concentrations of 100 mg/L.

In RPMI, high concentrations of AmB (15, 30 mg/L) induce hemolysis. PEGHS15 loaded in LNPs prevents this hemolytic effect of AmB at a concentration of 800 mg/L.

### Example 2: Composition Comprising Lipid Nanoparticles Comprising AmB and PEG15HS (LNPs)

### 2.1. Material and Methods

Formulations comprising AmB and PEG15HS incorporated in lipid nanoparticles (LNPs) were prepared and assayed.

LNPs comprising increasing concentrations of PEG15HS were prepared as detailed above. The ingredients are detailed in Table 2.

TABLE 2
composition of LNPs

| | LNP-008 (LNP) | | LNP-009 (LNP + CH) | | LNP-014 (LNP + CH) | |
|---|---|---|---|---|---|---|
| Oily phase | | | | | | |
| Labrafac lipophile | 1000 | mg | 700 | mg | 700 | mg |
| WL 1349 | | | | | | |
| Phospholipon | 75 | mg | 100 | mg | 100 | mg |
| Cholesterol | 0 | | 200 | mg | 200 | mg |
| AmB | 2 mg | | | | | |
| Aqueous phase | | | | | | |
| Kolliphor HS15 | 850 | mg | 850 | mg | 850 | mg |
| NaCl | 90 | mg | 90 | mg | 90 | mg |
| Water | 2.9 | g | 2.9 | g | 2.9 | g |

### 2.2. Results

### 2.2.1. AmB Content in the LNPs

To determine the AmB content in the LNPs, 50 μL of LNP suspension was solubilized in 950 μL of DMSO. The absorbance of these solutions was measured at a wavelength of 407 nm using an Infinite M200 Pro microplate reader (Tecan®, France). The concentration of AmB in these solutions was estimated according to standard curves of free AmB in DMSO with concentrations ranging from (10 to 0.25 mg/L). Quality control solutions at 8, 2 and 0.4 mg/L were used to validate the assay). AmB concentrations (μg/ml or mg/L) of the solutions obtained by mixing 50 μL of freshly prepared LNP suspension with 950 μL DMSO are reported in Table 3 below.

### 2.2.2. Size Distribution and PDI

Size distributions of LNPs were determined by dynamic light scattering measurements using a Malvern NanoZS (Malvern, Orsay, France). LNP suspensions were diluted (1:60 v/v) with MilliQ water and analyzed in triplicate at 25° C. LNPs were characterized by the median value of the intensity-weighted size distribution and by the polydispersity index (PDI), which is a measure of the broadness of a size distribution. PDI is a number calculated from a two-parameter fit to the correlation data (the cumulants analysis). This index is dimensionless and scaled such that values smaller than 0.05 are mainly seen with highly monodisperse standards. PDI values bigger than 0.7 indicate that the sample has a very broad particle size distribution and is probably not suitable to be analysed by the dynamic light scattering (DLS) technique. Different size distribution algorithms work with data that fall between these two extreme values of PDI (i.e., 0.05-0.7). The calculations used for the determination of size and PDI parameters are defined in the ISO standard documents 13321:1996 E and ISO 22412:2008

The results are shown in Table 3 below.

TABLE 3
Size distribution and PDI of the LNPs
depending on their composition

| | LNP-008 | LNP-009 |
|---|---|---|
| | (LNP) | (LNP + CH) |
| [AMB] (mg/L) | 54 ± 10 | 68 ± 5 |
| Size (nm) | 84 ± 2 | 57 ± 1 |
| PDI | 0.197 | 0.214 |

### 2.2.2. Hemolytic Activity

Human blood samples from different healthy volunteers was collected in EDTA tubes and centrifuged at 3000×g for 5 minutes. Plasma was removed and the erythrocytes were washed four times with 2 ml of 0.9% NaCl by centrifugation at 3000×g for 5 minutes.

Erythrocytes were suspended in PBS pH 7.4 and their concentration was adjusted to 5.108 cells/mL. Erythrocytes were then incubated with 15 mg/L of AmB solubilised as a solution or loaded in LNP made of PEG15HS (LNP-008, LNP-009, LNP014) or made of other surfactants (LNP-015 and LNP-016) for 1 hour at 37° C. under shaking, then centrifuged at 3000×g for 5 minutes. Then, 100 μL of the supernatant were transferred in a flat-bottomed transparent 96-well plate (SARSTEDT, France) and the absorbance was measured at 540 nm using a plate reader (Infinite M200 PRO, Tecan®, France). Untreated erythrocytes dispersed in PBS were used as a negative control, and erythrocytes treated by 1% (v/v) of TritonX-100 were used as a positive control. Percentage of hemolysis was calculated using the following equation: 100*(Asample−Acontrol)/(aTritonX-100−Anegative), where Asample is the absorbance measured for each experimental conditions, Anegative and aTritonX-100 are the absorbance of negative and positive controls, respectively.

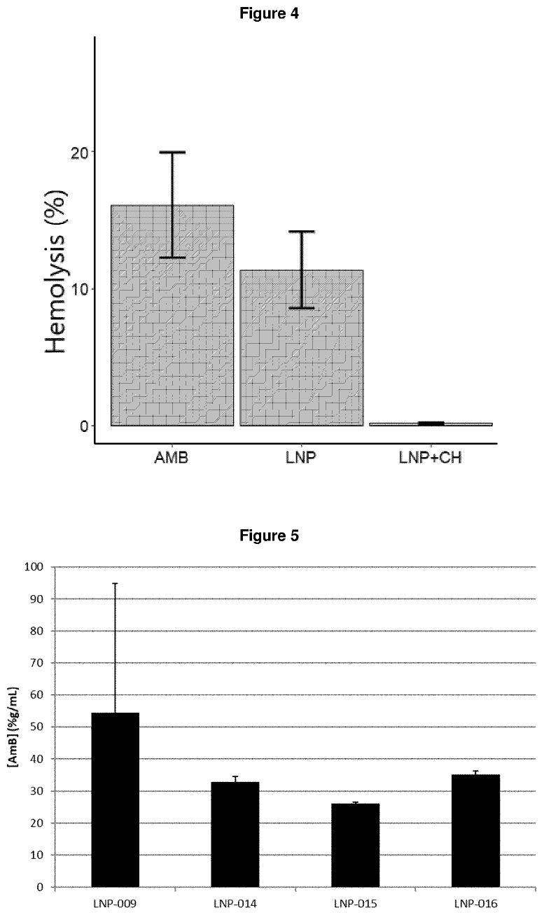

The results are presented in FIG. 4.

### Comparative Example 3: Compositions Comprising Lipid Nanoparticles Comprising AmB and Other Surfactants that PEG15HS

### 3.1. Material and Methods

LNPs corresponding to LNP-009 according to Example 2 with PEG15HS replaced by Brij® S20 and Brij® O20 are prepared with the ingredients detailed in Table 5 below.

TABLE 5

| | | | | | LNP-015 | | LNP-016 | |
|---|---|---|---|---|---|---|---|---|
| | LNP-009 | | LNP-014 | | (not according | | (not according to | |
| | (LNP + CH) | | (LNP + CH) | | to the invention) | | the invention) | |
| Oily phase | | | | | | | | |
| Labrafac | 700 | mg | 700 | mg | 700 | mg | 700 | mg |
| lipophile | | | | | | | | |
| WL 1349 | | | | | | | | |
| Phospholipon | 100 | mg | 100 | mg | 100 | mg | 100 | mg |
| Cholesterol | 200 | mg | 200 | mg | 200 | mg | 200 | mg |
| AmB | 4 mL of a solution of AmB in Chloroform 0.5 mg/mL | | | | | | | |
| Aqueous phase | | | | | | | | |
| Kolliphor HS15 | 850 | mg | 850 | mg | 0 | | 0 | |
| Brij ® S20 | 0 | | 0 | | 850 | mg | 0 | |
| Brij ® O20 | 0 | | 0 | | 0 | | 850 | mg |
| NaCl | 90 | mg | 90 | mg | 90 | mg | 90 | mg |
| Water | 2.9 | g | 2.9 | g | 2.9 | g | 2.9 | g |

### 3.2. Results

The results presented in FIG. 5 show that the AmB concentration in LNPs comprising AmB and PEG15HS (LNP-009 and LNP-014) is greater than or equivalent to that of LNPs comprising AmB and Brij® S20 (LNP-015) or Brij® O20 (LNP-016).

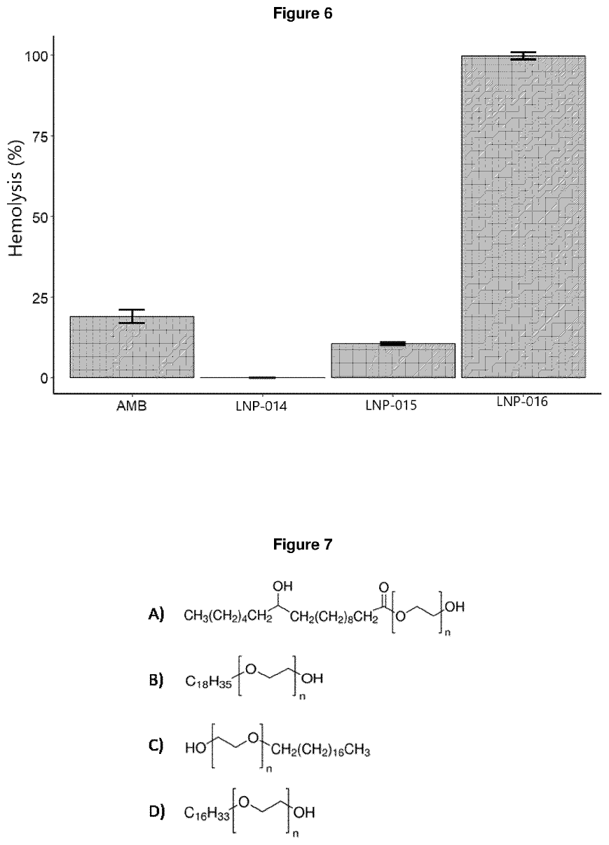

The results presented in FIG. 6 show that the hemolytic activity of LNPs comprising AmB and PEG15HS (LNP-014) is significantly lower than LNPs comprising AmB and Brij® S20 (LNP-015) or Brij® O20 (LNP-016).

### Comparative Example 4: Formulation Containing AmB and Various Surfactants Dispersed in Aqueous Phase (not According to the Invention)

### 4.1. Material and Methods

### 4.1.1. Strains

Twelve clinical isolates of Mucorales identified by ARNr sequencing were used: Rhizopus arrhizus (5), Rhizopus microsporus (1), Lichtheimia corymbifera (3), Lichtheimia ramosa (1), Mucor circinelloides (1), Rhizomucor pusillus (1). Isolates were subcultured from frozen stocks (−80° C.) on 75 cm³ flasks containing 15 mL of Sabouraud dextrose agar (Sigma-Aldrich, St. Quentin Fallavier, France) during 3 days at 37° C. Spores were collected by flooding the flask with 10 ml of sterile water. The suspensions were filtered on nylon filter (Millipore, Carrigtwohill, Ireland) with pore size of 11 μm to remove hyphal elements. The concentration of the spore suspensions was then adjusted in sterile water to obtain inocula of 2×105 spores/mL.

### 4.1.2. Media

RPMI 1640 (with L-glutamine and a pH indicator but without bicarbonate) (Sigma-Aldrich) was supplemented with dextrose to a final concentration of 2% (w/v) and buffered to pH 7 with 0.165 mol/L of MOPS (Sigma-Aldrich). This medium was sterilized using 0.22 μm pore-size filter. Phosphate buffered saline (PBS) at 0.05 mol/L having a pH of 7.4 was also used as medium to measure the hemolytic activity of AmB and UV spectra recording.

### 4.1.3. Preparation of Solutions

AmB powder (Sigma-Aldrich) was dissolved in dimethyl sulfoxide to get a stock solution at 8192 mg/L stored at −80° C. for six months. On the day of use, AmB and freshly made surfactant solutions (in RPMI or PBS) were diluted in the same medium (RPMI or PBS) to obtain desired concentrations. Surfactants used (polyethylene glycol (15)-hydroxystearate (also named Solutol® HS15 or Kolliphor® HS 15, or Macrogol 15 Hydroxystearate); polyoxyethylene (10) stearyl ether (Brij® S10); polyoxyethylene (20) stearyl ether (Brij® S20); polyoxyethylene (20) oleyl ether (Brij® 020); and polyoxyethylene (10) cetyl ether (Brij® C10)), which have their chemical structure presented in FIG. 7, were purchased from Sigma-Aldrich.

### 4.1.4. Checkerboard Assays

AmB minimum inhibitory concentrations (MIC) of each strain of Mucorales was measured in RPMI the presence of different concentrations of surfactant as described by the EUCAST broth microdilution antifungal susceptibility test methodology with modification for the checkerboard procedure. Checkerboards were performed by adding 100 μL of two-fold dilutions of AmB (0.008 to 4 mg/L) and surfactant solutions (0.04 to 2752 mg/L) in 96 well plates. One hundred microliters of a spore suspension at a concentration of 105 spores/mL were added to the wells, and the plates were incubated for 24 h at 37° C. MICs were read visually after 24 h as the lowest concentrations of AmB that completely inhibited the growth of the fungal strain. Three experiments were performed for each strain.

An inhibitory Eₘₐₓ model (Equation 1) developed by Chauzy et al. (Chauzy, A.; Buyck, J.; de Jonge, B. L. M.; Marchand, S.; Grégoire, N.; Couet, W. Pharmacodynamic Modelling of β-Lactam/β-Lactamase Inhibitor Checkerboard Data: Illustration with Aztreonam-Avibactam. Clin. Microbiol. Infect. 2019, 25, 515.e1-515.e4, doi: 10.1016/j.cmi.2018.11.025) to evaluate the enhancing effect of non-antibiotics molecules on the efficacy of antibiotic against bacteria was used here to describe the average decrease in AmB MIC values (M̅I̅C̅_{surf}) against various Mucorales strains in relation to surfactant concentrations (C_{surf}).

$${\overline{MIC}}_{surf} = {MIC}_{0} - \frac{( {MIC}_{0} - {MIC}_{∞} ) * {C}_{surf}}{{EC}_{50} + {C}_{surf}} \qquad ( Equation 1 )$$

In equation 1, MIC₀ refers to the AmB MIC measured without surfactants and MIC_{∞} is the lowest AmB MIC measured in the presence of surfactant. The maximal ratio in AmB MIC decrease defined as maximal efficacy (Eₘₐₓ) was calculated as the ratio of MIC₀/MIC_{∞}. EC₅₀ is the concentration of surfactant producing 50% of the Eₘₐₓ and characterizes the potency of the surfactants. These parameters were evaluated using WinNonlin software (version 6.2, Certara, NJ, USA) as described by Chauzy et al.

### 4.1.5. Hemolytic Activity

Hemolytic activity of AmB in the presence of surfactants was measured according to the protocols described in Serrano et al (Serrano, D. R.; Hernández, L.; Fleire, L.; González-Alvarez, I.; Montoya, A.; Ballesteros, M. P.; Dea-Ayuela, M. A.; Miró, G.; Bolás-Fernández, F.; Torrado, J. J. Hemolytic and Pharmacokinetic Studies of Liposomal and Particulate Amphotericin B Formulations. Int J Pharm 2013, 447, 38-46, doi: 10.1016/j.ijpharm.2013.02.038).

Human blood samples from different healthy volunteers was collected in EDTA tubes and centrifuged at 3000×g for 5 minutes. Plasma was removed and the erythrocytes were washed four times with 2 ml of 0.9% NaCl by centrifugation at 3000×g for 5 minutes.

Erythrocytes were suspended in RPMI or PBS and their concentration was adjusted to 5.108 cells/mL. Erythrocytes were then incubated with different combinations of AmB (0.5 mg/L) and surfactants (0, 5, 10, 20, 50, 100 mg/L) for 1 hour at 37° C. under shaking, then centrifuged at 3000×g for 5 minutes. Then, 100 μL of the supernatant were transferred in a flat-bottomed transparent 96-well plate (SARSTEDT, France) and the absorbance was measured at 540 nm using a plate reader (Infinite M200 PRO, Tecan®, France). Untreated erythrocytes dispersed in RPMI or PBS were used as a negative control, and erythrocytes treated by 1% (v/v) of TritonX-100 were used as a positive control. Percentage of hemolysis was calculated using the following equation: 100*(Aₛₐₘₚₗₑ₋−A_{control})/(A_{TritonX-100}−A_{negative}), where Aₛₐₘₚₗₑ is the absorbance measured for each experimental conditions, Anegative and ATritonX-100 are the absorbance of negative and positive controls, respectively. Experiments were performed in duplicate. After these preliminary experiments, experiments were repeated with combinations of AmB (0, 1, 5, 15 mg/L) and Solutol® HS15 (0, 5, 10, 20, 50, 100, 200, 400 mg/L) in PBS and combinations of AmB (0, 15, 30, 45 and 60 mg/L) and Solutol® HS15 (0, 5, 10, 20, 50, 100, 200, 400 mg/L) in RPMI. The experiments were performed in duplicate on four experiments for RPMI and in duplicate on two experiments for PBS.

### 4.2. Results

### 4.2.1. Efficacy of PEG15HS and Other Surfactants to Improve the Efficacy of AmB in Solution Against Mucorales

The efficacy of PEG15HS to improve the efficacy of AmB against several genera and strains of Mucorales involved in mucormycosis was assayed.

All isolates had a MIC for AmB less than 1 mg/L, while MIC values greater than 1024 mg/L were obtained for Solutol® HS15 alone. Under this condition, fractional inhibitory concentration (FIC) index analysis, traditionally used to assess the efficacy of combinations of antimicrobial agents, can difficultly be used to evaluate the influence of Solutol® HS15 on AmB efficacy.

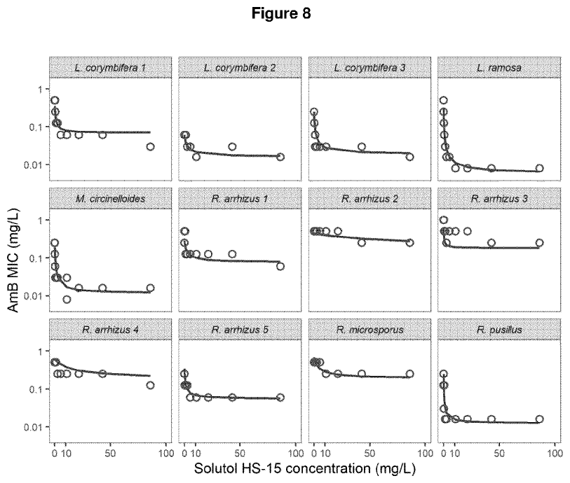

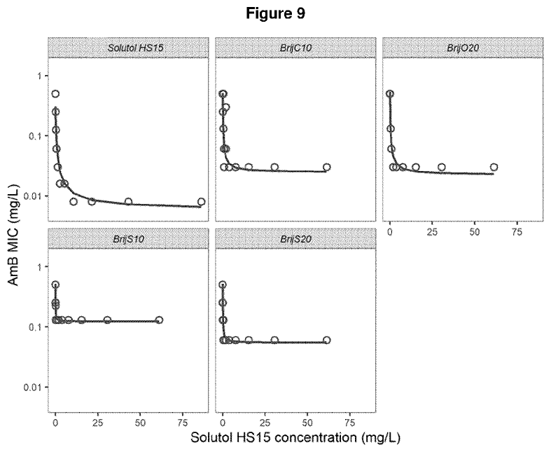

In addition, the decrease in the MIC of AmB as a function of Solutol® HS15 concentrations exhibited exponential decay profiles (FIG. 8).

The curves of FIG. 8 were further analyzed with an inhibitory Eₘₐₓ model, and the maximum efficacy (Eₘₐₓ) and potency (EC₅₀) of Solutol® HS15 to decrease the MIC of AmB against the different strains were used as comparison variables. The results are shown in Table 4.

TABLE 4
Parameter estimated by the inhibitory Emax model for the
strains of Mucorales in the presence of Solutol@
HS15 or different Brij ® surfactants. MIC0: MIC of AmB in the
absence of surfactant. EC50: surfactant concentration producing 50% of
Emax. MIC_{∞}: MIC_{∞} is the lowest AmB MIC measured
in the presence of surfactant.

| | | | | | Eₘₐₓ |
|---|---|---|---|---|---|
| | | MIC₀ | EC₅₀ | MIC_{∞} | (MIC0/ |
| Surfactant | Isolates | (mg/L) | (mg/L) | (mg/L) | MIC_{∞}) |
| Solutol ® | L. corymbifera 1 | 0.36 | 0.44 | 0.068 | 5.2 |
| HS15 | | | | | |
| | L. corymbifera 2 | 0.06 | 1.33 | 0.016 | 3.8 |
| | L. corymbifera 3 | 0.25 | 0.42 | 0.019 | 13.0 |
| | L. ramosa | 0.51 | 0.19 | 0.008 | 63.8 |
| | M. circinelloides | 0.19 | 0.32 | 0.012 | 16.5 |
| | R. arrhizus 1 | 0.28 | 1.34 | 0.077 | 3.7 |
| | R. arrhizus 2 | 0.44 | 53.45 | 0.177 | 2.5 |
| | R. arrhizus 3 | 0.59 | 0.28 | 0.183 | 3.2 |
| | R. arrhizus 4 | 0.61 | 8.57 | 0.186 | 3.3 |
| | R. arrhizus 5 | 0.35 | 0.47 | 0.055 | 6.4 |
| | R. microsporus | 0.68 | 2.32 | 0.190 | 3.6 |
| | R. pusillus | 0.25 | 0.13 | 0.012 | 19.9 |
| Brij ® S10 | L. ramosa | 0.51 | 0.02 | 0.123 | 4.1 |
| Brij ® S20 | L. ramosa | 0.51 | 0.02 | 0.056 | 9.2 |
| Brij ® O20 | L. ramosa | 0.51 | 0.06 | 0.022 | 23.2 |
| Brij ® C10 | L. ramosa | 0.51 | 0.05 | 0.025 | 20.4 |

The Eₘₐₓ of Solutol® HS15 to improve the antifungal action of AmB varied from 2.5 to 63.8 depending on the isolate (Table 1). The highest value was obtained for the species Lichtheimia ramosa. Eₘₐₓ values were also relatively high against Lichtheimia corymbifera species, expect for one strain (Lichtheimia corymbifera 2), but the efficacy of AmB alone against this strain was already high, as shown by its low MIC₀ value of 0.06 mg/L. Thus, it seems difficult to further reduce the already low MIC of AmB against this strain. Against the tested strains of Rhizomucor pusillus and Mucor circinelloides, the combination AmB-Solutol® HS15 was also effective, with an Eₘₐₓ of 20 and 16.5, respectively. However, for the 2 species of the genus Rhizopus tested (one microsporus and 5 strains of arrhizus (formerly oryzae)), the combination seemed less effective, with Eₘₐₓ values below 4, with the exception of one strain of arrhizus, which had an Eₘₐₓ value of 6.4.

When Eₘₐₓ values were above 4, Solutol® HS15 was generally very potent, with EC₅₀ values below 0.5 mg/L, showing that Solutol® HS15 improved AmB efficacy at low concentrations. When Eₘₐₓ was less than 4, as for most of Rhizopus strains, EC₅₀ values were mainly above 1 mg/L, and could go up to 53.45 mg/L.

The results obtained with Solutol® HS15 showed that this surfactant was able to improve by more than 4 times the efficacy of AmB against several strains of Mucorales at low concentrations.

To assess whether this effect was specific for Solutol® HS15 or could also be obtained with other nonionic polyethoxylated surfactants, the antifungal efficacy of AmB associated with different Brij® was also tested against L. ramosa, the strain of Mucorales that responded the most to the AmB-Solutol® combination. The Brij® surfactants tested had variable chain lengths for the hydrophobic (acyl group) and hydrophilic (polyethylene oxide) parts (FIG. 7).

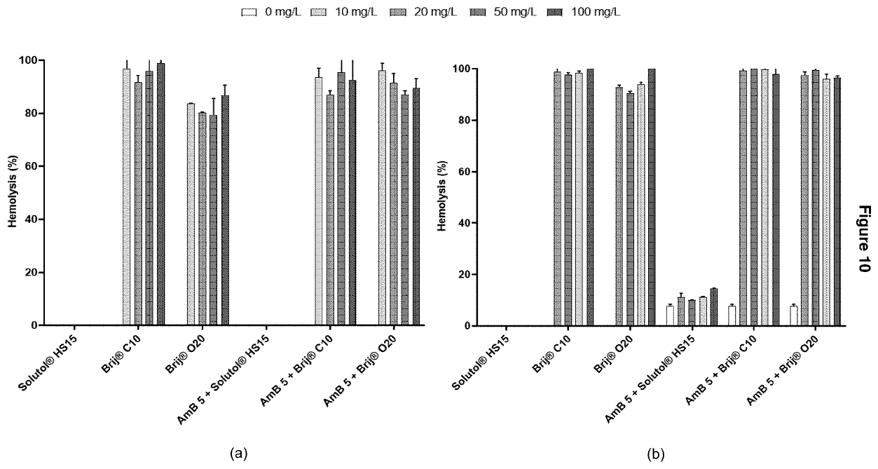

The results presented in FIG. 9 show that Brij® S10, Brij® S20, Brij® O20 and Brij® C10 were also able to increase the efficacy of AmB on L. ramosa. The four nonionic surfactants tested showed high potencies with EC₅₀ values above 0.1 mg/L (Table 1). However, while Brij® O20 and Brij® C10 presented efficacy close to that of Solutol® HS15 (23.27 and 20.4 respectively), Brij® S10 and Brij® S20 were less efficient with efficacy of 4.1 and 9.2 respectively (Table 4).

### 4.2.2. Hemolytic Activity

The results presented above have shown that certain nonionic polyethoxylated surfactants, mainly Solutol® HS15, Brij® O20 and Brij® C10 were able to potentiate the antifungal activity of AmB against various isolates of Mucorales. However, these results are only interesting if the potential human cytotoxicity of AmB is not also increased and the combinations have favourable benefit/risk ratios. Indeed permeabilisation of the mammalian cell membrane by AmB is a potential adverse effect that could also be enhanced by these nonionic surfactants.

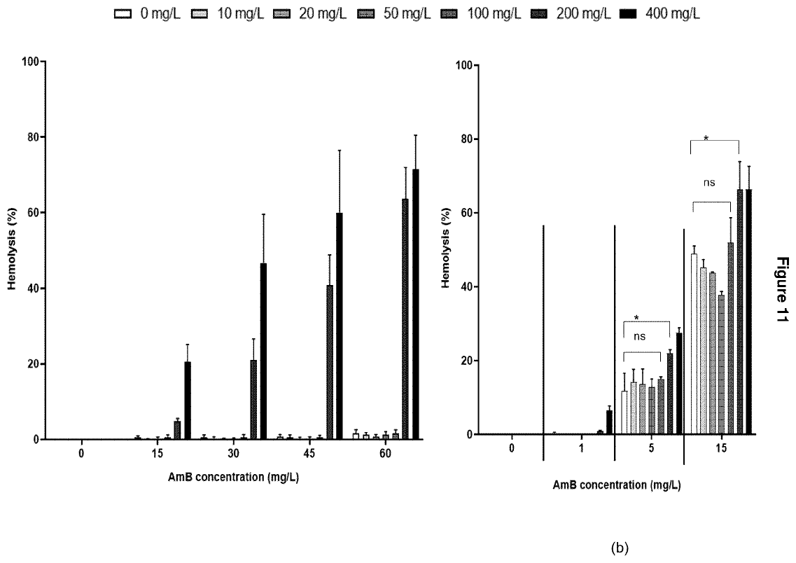

To evaluate this potential adverse effect, the hemolytic activity of 5 mg/L of AmB alone or in the presence of various concentrations of Solutol® HS15, Brij® O20 and Brij® C10 (0, 10, 20, 50 and 100 mg/L) was measured on human erythrocytes dispersed in RPMI (FIG. 10a) or in PBS (FIG. 10b). The results are shown in FIG. 10.

Interestingly, Solutol® HS15 alone was not hemolytic in the concentration range tested in both media, while Brij® O20 and Brij® C10 alone were both highly hemolytic (greater than 90% hemolysis) from the lowest concentration tested (10 mg/L) in both media.

At 5 mg/L, AmB alone was not hemolytic in RPMI (FIG. 10a), whereas it hemolyzed 7.7±0.7% of erythrocytes when they were dispersed in PBS (FIG. 10b).

In RPMI and PBS, Solutol® HS15 at concentrations up to 100 mg/L, did not potentiate the hemolytic activity of AmB (FIG. 4).

Further hemolytic experiments were performed with different concentrations of AmB (1, 5, 15, 30, 45 and 60 mg/L) and Solutol® HS15 (0, 10, 20, 50, 100, 200 and 400 mg/L) in solution.

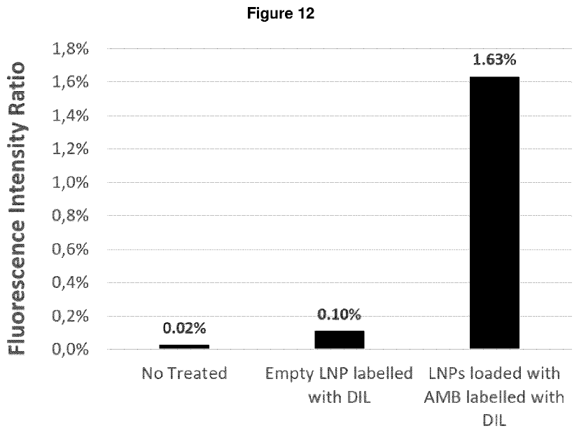

The results are shown in FIG. 11. FIG. 11a represents the results in RPMI. FIG. 11b represent the results in PBS.

For concentrations up to 100 mg/L, Solutol® HS15 did not increase the hemolytic activity of AmB regardless of the medium and AmB concentrations. However, for Solutol® HS15 concentrations of 200 and 400 mg/L, AmB hemolytic activity increased in both media. This increase in hemolytic activity was more spectacular in RPMI than in PBS. For example, for 15 mg/L of AmB, the hemolysis was of 0.6±0.6% in RPMI for 100 mg/L of Solutol® HS15 and increased to 20.5±4.7% for 400 mg/L of Solutol® HS15. In PBS, the hemolysis was of 52.0±6.7% in RPMI for 100 mg/L of Solutol® HS15 and increased to 66.4±6.3% for 400 mg/L of Solutol® HS15. Again, the hemolytic activity of AmB was much higher in PBS than in RPMI. For example, for 15 mg/L pure AmB, 49.0±2.1% hemolysis was observed in PBS, while less than 0.1% was obtained in RPMI at this AmB concentration.

### Example 5: Composition Comprising Lipid Nanoparticles (LNPs) Comprising AmB PEG15HS and Isavuconazole (ISA)

### 5.1. Material and Methods

Prior to the formulation of LNPs, the combination of AmB, ISA and PEG15HS in solution was evaluated.

AmB minimum inhibitory concentrations (MIC) of each strain of Mucorales was measured in RPMI in the presence of different concentrations of ISA or PEG15HS as described by the EUCAST broth microdilution antifungal susceptibility test methodology with modification for the checkerboard procedure. Checkerboards were performed by adding 100 μL of two-fold dilutions of AmB (0.008 to 4 mg/L) and ISA (0.008 to 4 mg/L) or PEG15HS (0.04 to 2752 mg/L) in 96 well plates. One hundred microliters of a spore suspension at a concentration of 105 spores/mL were added to the wells, and the plates were incubated for 24 h at 37° C. MICs were visually read after 24 h as the lowest concentrations of AmB that completely inhibited the growth of the fungal strain. Three experiments were performed for each strain.

AmB MIC of each strain of Mucorales was measured in RPMI in the presence of different concentrations of ISA and a constant concentration of PEG15HS (100 times the concentration of AmB) as described by the EUCAST broth microdilution antifungal susceptibility test methodology with modification for the checkerboard procedure. Checkerboards were performed by adding 100 μL of two-fold dilutions of AmB (0.008 to 4 mg/L)+PEG15HS (0.8 to 400 mg/L) and ISA (0.008 to 4 mg/L) in 96 well plates. One hundred microliters of a spore suspension at a concentration of 105 spores/mL were added to the wells, and the plates were incubated for 24 h at 37° C. MICs were visually read after 24 h as the lowest concentrations of AmB that completely inhibited the growth of the fungal strain. Three experiments were performed for each strain.

LNPs Preparation

Formulations comprising lipid nanoparticles (LNPs) loaded with AmB+PEG15HS and LNPs loaded AmB+PEG15HS+ISA were prepared as follows:

Briefly, a mass (80-120 mg) of an oil phase consisting of triglycerides (Labrafac®, or tributyrin in different mass ratios, i.e. 100% Labrafac® to 100% tributyrin) was supplemented with AmB (0,025 to 0.2 mg) and ISA (0.25 to 5 mg) and then mixed with 80 to 120 mg of PEG15HS (Solutol HS15®) and 500 to 800 μl of water to form an oil-in-water emulsion. This emulsion was then heated to 85° C., to obtain a water-in-oil emulsion, and then cooled to 40° C. Three such temperature cycles were performed and the system was then fast cooled to 4° C. The formulated nanoparticles were then characterized by size distribution and mean zeta potential measurements. Examples of formulations of LNPs loaded with AmB and AmB-ISA are detailed in Table 5:

TABLE 5
Composition of AmB-loaded (nano AmB) and
AmB-ISA-loaded (nano AmB + ISA) LNPs

| | mass | mass | mass | mass | total oil | Mass | Volume |
|---|---|---|---|---|---|---|---|
| Formulation | ISA | AmB | Labrafac | Tributyrin | mass | Solutol ® | water |
| number | (mg) | (mg) | (mg) | (mg) | (mg) | HS15 | (ml) |
| nano AmB | 0 | 0.05 | 40 | 40 | 80 | 120 | 800 |
| nano AmB + ISA | 2.5 | 0.025 | 40 | 40 | 80 | 120 | 800 |

### Strains Used

Rhizopus arrhizus 1, Lichtheimia corymbifera and Lichtheimia ramosa strains were used. These correspond to the most frequently encountered species in human mucormycosis. These three strains were isolated from patients suffering from pulmonary mucormycosis.

Two reference strains (ATCC 16424 and ATCC 204305) and a clinical azole-resistant strain (AfR2) of Aspergillus fumigatus were used.

### 5.2. Results

5.2.1 Efficacy of LNPs Against Mucorales and Aspergillus fumigatus

Combination of AMB and ISA in solution was compared to the combination of AMB ISA and PEG15HS in solution. The results are presented in Table 6 below:

TABLE 6
MICs in solution

| | Rhizopus arrhizus 1 | L. corymbifera | L. ramosa |
|---|---|---|---|
| MIC of AmB (mg/L) | 1 | 1 | 1 |
| MIC of ISA (mg/L) | 1 | 4 | 4 |
| MIC of AmB c/* ISA | 0.125 | 0.25 | 0.25 |
| (mg/L) | (diminution of 8) | (diminution of 4) | (diminution of 4) |
| MIC of AmB c/* | 0.03 | 0.03 | 0.06 |
| PEG15HS (mg/L) | (diminution of 32) | (diminution of 32) | (diminution of 16) |
| MIC of AmB c/* | 0.008 | 0.008 | 0.008 |
| PEG15HS and ISA | (diminution of 125) | (diminution of 125) | (diminution of 125) |
| (mg/L) | | | |
| MIC of ISA c/* AmB | 0.125 | 0.125 | 0.125 |
| and PEG15HS (mg/L) | (diminution of 8) | (diminution of 32) | (diminution of 32) |

*c/: combined with
The results show that combination of ISA and AMB in solution decreased the MIC of AmB by a factor of 4 to 8. Combination of AmB and PEG15HS in solution decreased the MIC of AmB by a factor of 16 to 32. Combination of AmB, ISA and PEG15HS in solution decreased the MIC of AmB by a factor of 125. Combination of AmB, ISA and PEG15HS in solution decreased the MIC of ISA by a factor of 8 to 32.

### 5.2.2. Size Distribution and PDI

Size distributions of LNPs were determined by dynamic light scattering measurements using a Malvern NanoZS (Malvern, Orsay, France). LNP suspensions were diluted (1:60 v/v) with MilliQ water and analyzed in triplicate at 25° C. LNPs were characterized by the median value of the intensity-weighted size distribution and by the polydispersity index (PDI), which is a measure of the broadness of a size distribution. PDI is a number calculated from a two-parameter fit to the correlation data (the cumulants analysis). This index is dimensionless and scaled such that values smaller than 0.05 are mainly seen with highly monodisperse standards. PDI values bigger than 0.7 indicate that the sample has a very broad particle size distribution and is probably not suitable to be analysed by the dynamic light scattering (DLS) technique. Different size distribution algorithms work with data that fall between these two extreme values of PDI (i.e., 0.05-0.7). The calculations used for the determination of size and PDI parameters are defined in the ISO standard documents 13321:1996 E and ISO 22412:2008. The results are shown in Table 7 below:

TABLE 7
Size distribution and PDI of the LNPs depending on their composition

| Oil phase | Mass of Solutol ® (mg) | size (nm) average ± SD | PDI |
|---|---|---|---|
| Labrafac | 120 | 44.6 ± 1.6 | 0.13 ± 0.03 |
| | 90 | 141.5 ± 3.8 | 0.15 ± 0.02 |
| Tributyrin | 120 | 118.7 ± 11.4 | 0.16 ± 0.01 |
| | 80 | 222.6 ± 10.0 | 0.11 ± 0.10 |
| Labrafac/Tributyrin | 120 | 39.7 ± 0.4 | 0.06 ± 0.02 |
| (1/1; m/m) | 90 | 123.8 ± 6.9 | 0.13 ± 0.001 |
| Labrafac/Tributyrin | 120 | 62.0 ± 12.2 | 0.13 ± 0.01 |
| (1/3; m/m) | 90 | 186.4 ± 3.7 | 0.17 ± 0.06 |

5.2.3 Efficacy of LNPs Against Mucorales and Aspergillus fumigatus

MICs of AmB in solution, ISA in solution, AmB-loaded LNPs, and AmB-ISA-loaded LNPs were evaluated according to the European Committee on Antimicrobial Susceptibility Testing (EUCAST) guidelines. Briefly, MICs were determined by adding 100 μL of two-fold dilutions of AmB in solution, ISA in solution, AmB-LNPs or AmB-ISA-LNPs (0.008 to 4 mg/L) in 96-well plates. One hundred microliters of a spore suspension at a concentration of 105 spores/mL were added to the wells, and the plates were incubated for 24 h (Mucorales) or 48 h (A. fumigatus) at 37° C. MICs were read visually after 24 h as the lowest concentrations of AmB that completely inhibited the growth of the fungal strain. Three experiments were performed for each strain. The results are shown in Table 8 and Table 9:

TABLE 8
MICs for different Mucorales

| | Rhizopus arrhizus 1 | | | Lichtheimia corymbifera | | | Lichtheimia ramosa | | |
|---|---|---|---|---|---|---|---|---|---|
| | MIC | | | MIC | | | MIC | | |
| | (mg/L) | ratio | ratio | (mg/L) | ratio | ratio | (mg/L) | ratio | ratio |
| AmB | 1 | — | — | 1 | — | — | 1 | — | — |
| ISA | 1 | — | — | 4 | — | — | 4 | — | — |
| Nano AmB | 0.03 | 33.33 | — | 0.03 | 33.33 | — | 0.03 | 33.33 | — |
| Nano AmB + ISA | 0.08/0.8 | 125 | 1.25 | 0.016/1.5 | 62.5 | 2.67 | 0.03/3 | 33.33 | 1.33 |

TABLE 9
MICs for different Aspergillus fumigatus strains

| | Aspergillus fumigatus 16 | | | A. fumigatus 20 | | | A. fumigatus R2 | | |
|---|---|---|---|---|---|---|---|---|---|
| | MIC | Ratio | Ratio | MIC | | | MIC | | |
| | (mg/L) | (AmB) | (ISA) | (mg/L) | ratio | ratio | (mg/L) | ratio | ratio |
| AmB | 2 | — | — | 2 | — | — | 2 | — | — |
| ISA | 0.5 | — | — | 0.5 | — | — | >4 | — | — |
| Nano AmB | 0.06 | 33 | — | 0.125 | 16 | — | 0.125 | 16 | — |
| Nano AmB + ISA | 0.04/0.2 | 500 | 2.5 | 0.002/0.1 | 1000 | 5 | 0.5/25 | 4 | — |

The results show that formulating AmB in nanoparticles comprising SOLUTOL H15S® improve the effect of AmB by a factor of 33 compared to that observed in solution against the main strains of Mucorales responsible for pulmonary infection (Table 8). These results are also found against Aspergillus fumigatus strains sensitive and resistant to azole antifungals responsible for pulmonary infection (Table 9). In Table 8 and Table 9 above, “Ratio (AmB)” refers to the ratio of the MIC of AmB alone to the MIC of AmB in the nanoparticle and “Ratio (ISA)” refers the ratio of the MIC of ISA alone to the MIC of ISA in the nanoparticle.

The presence of ISA strongly reinforces this gain in efficiency by a factor of 125 to 500 compared to that of AmB in solution.

### Example 6: Efficacy of Composition Comprising Lipid Nanoparticles Comprising AmB PEG 15HS (LNPs) Towards C. neoformans

Cryptococcus neoformans is a yeast (fungus) involved in life-threatening invasive infections worldwide named cryptococcosis. C. neoformans is responsible for meningoencephalitis, mainly in immunocompromised patients, leading to approximately 200 000 deaths each year. This yeast therefore represents a major public health problem worldwide.

The combination of AmB and 5-fluorocytosine is currently the recommended first-line treatment. Two formulations of AmB are mainly used, AmB deoxycholate (AmBd) and liposomal AmB (L-AmB). L-AmB is the less toxic form and is preferred in developed countries. However, it is the much more expensive form and is consequently not widely used in developing countries. AmBd represents the cornerstone of the management of cryptococcosis in developing countries. Unfortunately, the efficacy of the treatment against Cryptococcus remains low with 35-40% of mortality both in in resource-rich and resource-poor settings. In this context, there is an urgent need for new treatments.

Repurposing of existing drugs and improving efficacy of existing antifungal agents is a priority way of research. As demonstrated in the Examples above, polyethylene glycol (15)-hydroxystearate (PEG15HS), a non-ionic surfactant, can improve AmB efficacy against Mucorales and Aspergillus fumigatus. This combination reduces the MIC of AmB by up to 60-fold against Mucorales. Since PEH15HS is a FDA-validated surfactant for parenteral use, the combination of AmB and PEG15HS could be use against invasive fungal infection.

Lipid nanoparticles (LNPs) are water-dispersible lipid systems that allow combination of multiple molecule. The formulation of AmB and PEG15HS in LNPs could allow a homogeneous distribution of both compounds and potentially an intracellular action. Since AmB is the first line treatment of Cryptococcosis, combination of AmB and PEG15HS could be useful in Cryptococcosis. In addition, without wanting to be bound by a theory, the inventors believe that at industrial scale, preparing AmB and PEG15HS in LNPs would be much simple that of preparing L-AmB.

### 6.1. Material and Methods

Eight clinical strains of Cryptococcus neoformans were used, as shown in Table 10 below:

TABLE 10
Strains of Cryptococcus neoformans used

| Strains | Serotype | Site of infection |
|---|---|---|
| C1 | A | Lung (biopsy) |
| C2 | AD | CSF* |
| C3 | A | Blood |
| C4 | A | CSF* |
| C5 | A | Lung (BAL**) |
| C6 | | Tracheal aspiration |
| C7 | A | CSF* |
| C8 | A | Lung (BAL**) |

**BAL: broncho-alveolar lavage;
*CSF: cerebrospinal fluid;
Isolates were subcultured from frozen stocks (−80° C.) on Sabouraud dextrose agar (Sigma-Aldrich, St. Quentin Fallavier, France) over 48 h at 37° C. before use.

RPMI 1640 (with L-glutamine, pH indicator, no bicarbonate) (Sigma-Aldrich, Saint-Quentin-Fallavier, France) supplemented with 2% (w/v) dextrose and buffered to pH 7 with 0.165 mol/L MOPS (Sigma-Aldrich) was used for minimum inhibitory concentration (MIC) measurements. This medium was sterilized using a 0.22 μm pore-size filter.

AmB powder (Sigma-Aldrich) was dissolved in dimethyl sulfoxide to obtain a stock solution at 1000 mg/L, stored at −80° C. for six months.

On the day of use, AmB and freshly made AmB-loaded LNPs were diluted in RPMI medium to obtain the desired concentrations. The AmB-loaded LNPs were prepared as detailed in Example 5.

MICs of AmB alone and AmB formulated in LNPs were evaluated according to the European Committee on Antimicrobial Susceptibility Testing (EUCAST) guidelines. Briefly, MICs were determined by adding 100 μL of two-fold dilutions of AmB (0.008 to 4 mg/L) and LNPs-AmB (0.04 to 1024 mg/L) in 96-well plates. One hundred microliters of a fungal suspension at a final concentration of 105 CFU/mL was added to the wells, and the plates were incubated for 48 h at 30° C. MICs, defined as the lowest concentration of AmB inhibiting 90% of the growth of the fungal strain, were read after 24 hours of incubation by measuring absorbance at 530 nm with an Infinite M200 Pro microplate reader (Tecan®, France). Three experiments were performed for each strain.

### 6.2. Results/Discussion

All the isolates had an MIC for AmB within the range of 0.25-1 mg/L, while MIC values greater than 1 024 mg/L were obtained for blank-LNPs. MICs of AmB-loaded LNPs varied from 0.03 to 0.125 mg/L. The MIC reduction obtained with AmB-LNPs ranged from 8 to 32, depending on the isolate (Table 11). Furthermore, for concentrations below their MIC, AmB-loaded LNPs slowed the growth rate of the cryptococci tested, while AmB alone, at a concentration below its MIC, had no effect on the growth profile of the cryptococcal strains tested.

TABLE 11
MICs obtained for the Cryptococcus neoformans strains tested

| | MIC (mg/L) | | Diminution |
|---|---|---|---|
| Strain | AmB* | LNP-AmB** | of MIC |
| C1 | 0.5 | 0.03 | 16 |
| C2 | 0.25 | 0.03 | 8 |
| C3 | 0.5 | 0.06 | 8 |
| C4 | 0.5 | 0.06 | 8 |
| C5 | 1 | 0.03 | 32 |
| C6 | 1 | 0.06 | 16 |
| C7 | 1 | 0.125 | 8 |
| C8 | 1 | 0.125 | 8 |

*AmB: amphotericin B;
**LNP: AmB-loaded lipid nanoparticles

### Example 7: AmB-Loaded Lipid Nanoparticles Penetrate Cryptococcus Cells More Effectively than Nanoparticles not Loaded with Amb

### 7.1. Material and Methods

Cryptococcus neoformans Staining

Prior to nanoparticles treatments, Cryptococcus neoformans were stained 45 minutes with 5 μg/ml CellTracker Green CMFDA, a stable, nontoxic fluorescent probe for living cells. Fungi were then seeded in 8-well chamber slides (ibidi, France) suitable for high-resolution fluorescent observation using an Olympus FluoView FV-3000 confocal laser scanning microscope.

### Nanoparticle Treatments

The fungi were then incubated with empty LNPs labelled with DIL (AmB-blank LNPs) or LNPs loaded with AmB labelled with DIL (AmB-loaded LNPs) for 18h. Non-treated fungi were used as reference.

Imaging of Cryptococcus neoformans by Confocal Laser Scanning Microscopy

Planktonic Cryptococcus neoformans were observed using a 100x oil immersion objective lens and sequentially excited at 488 and 555 nm to visualize CellTracker Green (excitation 488 nm-emission range: 500-540 nm) and DiL (excitation 555 nm-emission range: 570-670 nm) dyes. Twenty to thirty stacks of horizontal plane images (1024×1024 pixels corresponding to 127×127 μm) with a z-step of 0.5 μm were acquired for each treatment condition.

Three-dimensional projections of Cryptococcus neoformans were constructed using the Easy 3D function of the IMARIS software (Bitplane). During the creation of the surfaces, the same segmentation parameters (particle size, absolute intensity and lack of post-segmentation filters) were applied to all 3D constructions whatever the treatment.

Images taken by confocal laser scanning microscopy images 18 h after incubation were analyzed to determine the fluorescence intensity ratios between the Cryptococcus cells marker (CellTracker Green CMFDA) and the lipid nanoparticles marker (DiL).

### 7.2. Results

The results are shown on FIG. 12.

The analysis of confocal laser scanning microscopy images showed that, quite unexpectedly, AmB-loaded LNPs penetrated Cryptococcus cells 16 times more efficiently than AmB-blank LNPs (FIG. 12).

These data suggest that AmB-loaded LNPs could enhance the entry of other antifungals (especially lipophilic ones, in particular azoles such as isavuconazole) into fungal cells. Indeed, antifungals such as azoles (including isavuconazole) are substrates of membrane efflux pumps, which decreases their efficiency by decreasing their intracellular concentration. The use of AmB-loaded LNPs could allow to bypass the effect of these pumps, resulting in higher isavuconazole intracellular concentration. It is known that efflux proteins play a role in azole extrusion and tolerance i.e. in fungal resistance to azoles. Thus, AmB-loaded LNPs could be of use in overcoming antifungal azole resistance by improving their delivery into fungal cells, thereby improving the efficiency of such antifungals.

1. A composition comprising an aqueous phase, a polyene antifungal and lipid nanoparticles which are dispersed in the aqueous phase, said lipid nanoparticles comprising an oily internal phase and an envelope, the envelope comprising a polyglycol ester of a hydroxy fatty acid, the polyene antifungal being dispersed in the aqueous phase and/or being part of the lipid nanoparticles.

2. Composition according to claim 1, wherein the hydroxy fatty acid is a C8-C24 hydroxy fatty acid.

3. Composition according to claim 1, wherein the polyglycol ester of a fatty acid comprises from 1 to 100 ethylene oxide groups.

4. Composition according to claim 1, wherein the polyglycol ester of a fatty acid comprises a C14-C18 hydroxy fatty acid.

5. Composition according to claim 1, wherein the polyglycol ester of a hydroxy fatty acid is polyethylene glycol-15-hydroxystearate.

6. Composition according to claim 1, wherein the polyene antifungal is selected from amphotericin B, nystatin, partricin, natamycin or a mixture thereof.

7. Composition according to claim 1, comprising between 1 g/L and 5 g/L of polyene antifungal and between 20 g/L and 100 g/L of polyglycol ester of a hydroxy fatty acid.

8. Composition according to claim 1, comprising between 30 mg/L and 120 mg/L of polyene antifungal and between 600 mg/L and 2400 mg/L of a hydroxy fatty acid.

9. Composition according to claim 1, wherein the lipid nanoparticles have a size preferably comprised between 1 and 250 mm.

10. Composition according to claim 1, wherein the lipid nanoparticles are free of antifungal polyene.

11. Composition according to claim 1, wherein the aqueous phase is free of polyene antifungal.

12. Composition according to claim 1, wherein the lipid nanoparticles comprise the polyglycol ester of a hydroxy fatty acid and the polyene antifungal in a weight ratio polyglycol ester of hydroxy fatty acid:polyene antifungal ranging from 5:1 to 100:1.

13. Composition according to claim 1, wherein the composition further comprises a triazole antifungal or an antifungal subject to fungal resistance, wherein the triazole antifungal or the antifungal subject to fungal resistance is dispersed in the aqueous phase and/or is part of the lipid nanoparticles.

14. Composition according to claim 13, wherein the triazole antifungal is selected from isavuconazole, itraconazole, posaconazole, voriconazole, fluconazole and a mixture thereof.

15. Lipid nanoparticles comprising an antifungal polyene, an oily internal phase and an envelope, the envelope comprising a polyglycol ester of a hydroxy fatty acid and the polyene antifungal being part of the lipid nanoparticles.

16. Lipid nanoparticles according to claim 15, wherein the lipid nanoparticles further comprise a triazole antifungal or an antifungal subject to fungal resistance wherein the triazole antifungal or the antifungal subject to fungal resistance is part of the lipid nanoparticles.

17. A process to prepare the lipid nanoparticles according to claim 15, comprising:
a) providing an oily phase comprising at least a fatty substance and an antifungal polyene as defined above,
b) providing an aqueous phase comprising a polyglycol ester of a hydroxy fatty acid,
c) mixing the oily phase of step a) and the aqueous phase of step b),
d) cooling the mixture obtained in step c) to obtain the lipid nanoparticles.

18. The process according to claim 17, with the condition that when the lipid nanoparticles comprise a triazole antifungal or an antifungal subject to fungal resistance, the oily phase provided in step a) further comprises a triazole antifungal or an antifungal subject to fungal resistance.

19. Method for the prevention or treatment of an infection caused by a fungus comprising administering a composition according to claim 1, or lipid nanoparticles comprising an antifungal polyene, an oily internal phase and an envelope, the envelope comprising a polyglycol ester of a hydroxy fatty acid and the polyene antifungal being part of the lipid nanoparticles.

20. Method according to claim 19, wherein the fungus is of the Mucorales order.
